(19) United States
(12) Patent Application Publication
(10) Pub. No.: US 2011/0184293 A1
(43) Pub. Date: Jul. 28, 2011

(54) DEVICE, METHOD AND KIT FOR IN VIVO DETECTION OF A BIOMARKER

(76) Inventors: Elisha Rabinovitz, Haifa (IL); Abraham Rubinstein, Jerusalem (IL); Yechezkel Barenholz, Jerusalem (IL); Elena Khazanov, Beit Shemesh (IL); Abdel Kareem Azab, Ara (IL); Noam Emmanuel, Jerusalem (IL); Eylon Yavin, Jerusalem (IL)

(21) Appl. No.: 13/003,329

(22) PCT Filed: Jul. 9, 2009
(86) PCT No.: PCT/IL09/00691

§ 371 Date: Apr. 14, 2011

Related U.S. Application Data
(60) Provisional application No. 61/079,571, filed on Jul. 10, 2008.

(51) Int. Cl. A61B 6/00; A61K 49/00; C12N 11/14; C07K 17/14

(52) U.S. Cl. 600/476; 424/9.1; 435/176; 530/387.1

(57) ABSTRACT

The invention relates to a device and a system for in-vivo detection of a biomarker in the gastrointestinal tract. The invention further relates to a method for the in-vivo detection of a biomarker in the gastrointestinal tract such as e.g., the α1-antitrypsin precursor (A1AT biomarker), by using the recognition factor, e.g., trypsin immobilized to a solid surface. The invention further relates to a kit for the in-vivo detection of a biomarker in the gastrointestinal system.

### FIELD OF THE INVENTION

The invention relates to a device and a system for in-vivo detection of a biomarker in the gastrointestinal tract. The invention further relates to a method for the in-vivo detection of a biomarker in the gastrointestinal tract such as e.g., the α1-antitrypsin precursor (A1AT biomarker), by using the recognition factor, e.g., trypsin immobilized to a solid surface. The invention further relates to a kit for the in-vivo detection of a biomarker in the gastrointestinal system.

### BACKGROUND OF THE INVENTION

Early diagnosis of various diseases, including gastric carcinoma, a leading cause of cancer-related deaths worldwide, is crucial for maximizing medical treatment efficacy of the disease. It is known that certain biomarkers expressed in the gastric juice are a sign of gastric cancer. One example of such a biomarker is human α1-antitrypsin precursor (A1AT), a 52 Kd member of the serine protease inhibitors (serpins family). Other examples for such a biomarker are CEA, and CA-19-9.

Although believed to be highly specific to neutrophil elastase, A1AT is a broad-spectrum protease inhibitor for many serine proteinases, including trypsin. Its proteolytic activity involves cleavage between Met³⁵⁸ and Ser³⁵⁹, which induces a conformational change of A1AT, locking the enzyme and its substrate into a stable, inactive 1:1 enzyme-inhibitor complex. The gastric juice of patients having early and advanced gastric cancer has been found to contain high levels of A1AT.

Kits for vitro testing of body fluid samples for the presence of a suspected substance are known in the art. For example, in some cases, diseases, such as cancer, are detected by analyzing blood samples for tumor specific markers, typically, proteins and nucleic acids. A drawback of this method is that the appearance of biomarkers in the blood stream usually occurs at a late stage of the disease, such that early detection is not possible using this method. Moreover, many of the biomarkers are common to several diseases and organs, and therefore their detection in the blood does not allow specific disease detection. Local detection of biomarkers in-vivo in the relevant organ might overcome such drawbacks and enable a sensitive and specific disease detection at early stages.

Currently, gastric cancer diagnosis requires invasive procedures that include upper GI endoscopy during which biopsies are taken and sent to pathology lab. Such procedures are invasive, time consuming and may miss-detect gastric cancer due to improper sampling. Thus, there is a need in the art for a non-invasive method for detecting gastric cancer at an early stage.

The development of orally administered wireless video pills (e.g. the PillCam® capsule, Given Imaging Ltd., Yokneam, Israel) for visualization of gastrointestinal (GI) epithelium injuries has simplified early in-vivo diagnosis of upper GI diseases. Typical examples for such visualization is the identification of mucosal lesions by monitoring structural as well as tone differences in color and fluorescence between injured and normal regions. Yet such visualization is not enough for conclusive diagnosis and does not eliminate the need for biopsy. Adding molecular detection of disease biomarkers found in the gastric fluids by the capsule can improve the diagnosis process and prevent the need for invasive endoscopy.

WO 09/057,120 describes a capsule that can sample intestinal fluids while traversing the gastrointestinal (GI) tract and may perform analysis of the sample for the presence of suspected substances onboard the capsule. Surpassingly, trying to immobilize the recognition factor directly onto the capsule surface, according to some of the embodiments described in WO 09/057,120, two significant drawbacks were discovered. The first one is poor surface density (number of recognition factors per unit surface) that can be achieved by techniques common in the art. This drawback may limit the sensitivity that can be achieved by the capsule. Another drawback refers to high non specific adsorption to the surface that may cause poor signal to noise ratio. One of the purposes of this invention is to overcome the above drawbacks.

### SUMMARY OF THE INVENTION

In one embodiment, the invention is a device for in-vivo detection of a cancer biomarker in the gastrointestinal system, the device comprising a housing. The housing may comprise an optical window and may enclose a detector or an imager and a light source. In some embodiments, an external surface of the optical window may be coated with a polymer. In some embodiments the polymer may have a recognition factor immobilized thereon via a spacer. In some embodiments, the imager may be configured to detect changes occurring at the optical window.

According to another embodiment, the invention is a device for in-vivo detection of a biomarker in the gastrointestinal system, the device comprising a housing. In some embodiments, the housing may comprise an optical window, a detector or an imager, a light source and a transparent slide. The transparent slide may be made of glass, silica, quartz, cellulose or any transparent plastic or polymer comprising a recognition factor immobilized onto the slide via a spacer. In some embodiments, a detector may be configured to detect optical changes on its surface. In other embodiments, an imager may be configured to image the transparent slide.

The invention is further directed to a system for in-vivo detection of a biomarker in the gastrointestinal system, the system comprising a device according to any of the embodiments detailed above and a transmitter to transmit images from the imager. The system may further comprise a receiving system to receive transmitted signals, and a display to display indication of the presence of a marker in-vivo.

The invention includes also a method for the in-vivo detection of the presence of a specific cancer biomarker in the gastrointestinal system of a subject comprising the step of orally administering a device, according to any one of the embodiments detailed above, to the subject. The method may further comprise the step of contacting the orally administered device with a detectable labeled binding agent that binds specifically to the biomarker or contacting the orally administered device with a first binding agent that binds specifically to the biomarker and a second detectable labeled binding agent that binds specifically to the first binding agent. In some embodiments, the presence of a bound label as detected by the imager may be indicative to the presence of said specific biomarker in the gastrointestinal system of the subject.

The invention further includes a diagnostic kit comprising a device according to any one of the embodiments detailed above, and a binding agent capable of specifically binding a biomarker which is labeled by a detectable label or a combination of a first binding agent that binds specifically to the biomarker and a second detectable labeled binding agent that binds specifically to the first binding agent. According to some embodiments, the binding agent or the combination may be either contained in a separate container or may be included in the device.

The invention is further directed to a transparent film coated by a recognition platform that binds to a specific biomarker. In some embodiments, the recognition platform may comprise a polymer and a recognition factor conjugated by a spacer.

The invention is further directed to a device for in-vivo detection of a biomarker in the gastrointestinal system, the device comprising a housing, said housing may comprise an optical window and may enclose a light receptor and a light source. In some embodiments, an external surface of the optical window may be coated by a polymer. The polymer may have a recognition factor immobilized thereon via a spacer. In some embodiments, the light receptor may be configured to detect light changes on the illuminated surface of the optical window.

In some embodiments of the present invention a device for in-vivo detection of a biomarker in the gastrointestinal system is provided. The device may comprise a housing. In some embodiments, the housing may comprise an optical window, a light receptor, a light source, and a glass slide. The glass slide may comprise a recognition factor immobilized onto the glass slide via a spacer. In some embodiments, the light receptor may be configured to detect light changes on the illuminated surface of the glass slide.

The invention is further directed to a system for in-vivo detection, the system comprising a device according to any one of the embodiments detailed above. In some embodiments the system may comprise a transmitter to transmit data from the light receptor. In some embodiments, the system may further comprise a receiving system to receive transmitted signals, and a display to display indication of the presence of a marker in-vivo.

The invention includes a method for the in-vivo detection of the presence of a specific cancer biomarker in the gastrointestinal system of a subject. The method may comprise the step of orally administering a device according to any one of the embodiments detailed above to the subject. In some embodiments the method may further comprise contacting the orally administered device with a detectable labeled binding agent that binds specifically to the biomarker or contacting the orally administered device with a first binding agent that binds specifically to the biomarker and a second detectable labeled binding agent that binds specifically to the first binding agent. In some embodiments, the presence of a bound label as detected by the light receptor may be indicative to the presence of the specific biomarker in the gastrointestinal system of the subject.

The invention includes a diagnostic kit comprising a device according to any one of the embodiments detailed above, and a binding agent capable of specifically binding a biomarker. In some embodiments, is the biomarker may be labeled by a detectable label or a combination of a first binding agent that binds specifically to biomarker and a second detectable labeled binding agent that binds specifically to the first binding agent; wherein the binding agent or the combination may be either contained in a separate container or may be enclosed in the device.

The invention includes a polycarbonate or a transparent film coated by a recognition platform that binds to a specific cancer biomarker. In some embodiments, the recognition platform may comprise a polymer having a recognition factor immobilized thereon via a spacer.

The invention further includes a glass slide that binds to a specific cancer biomarker. In some embodiments, the glass slide may comprise a recognition factor immobilized thereon via a spacer.

In an embodiment of the invention, there is provided a device for in-vivo detection of a biomarker in the gastrointestinal system, the device comprising: a housing comprising an optical window and enclosing a light receptor and a light source for illuminating in-vivo through the optical window; wherein an external surface of the optical window is coated by a polymer, the polymer having a recognition factor immobilized thereon via a spacer, and wherein the light receptor is configured to detect light changes on the illuminated surface of the optical window. The light receptor, in an embodiment of the invention, is a photodetector covered by high pass- or a notch filter configured to detect fluorescent changes on the illuminated surface of the optical window. The light source is one or more LED. In an embodiment of the invention, the light receptor is an imager or a CMOS.

In another embodiment, there is provided a device for in-vivo detection of a biomarker in the gastrointestinal system, the device comprising: a housing comprising an optical window, a light receptor and a light source for illuminating in-vivo and for illuminating the glass slide, and a glass slide comprising a recognition factor immobilized onto the glass slide via a spacer, wherein the light receptor is configured to detect light changes on the illuminated surface of the glass slide.

### BRIEF DESCRIPTION OF THE DRAWINGS

FIG. 1: Shows the chemical structures of the spacers: (A) LPEI (a) and LPEI-max (b), (B) O,O′-Bis[2-(N-Succinimidyl-succinylamino)ethyl]polyethylene glycol 3,000 (NHS-^{3K}PEG-NHS, (C) O—[(N-Succinimidyl)succinyl-aminoethyl]-O′-methylpolyethylene glycol 2,000 (NHS-^{2K}PEG).

FIG. 2: Depicts the grafting of spacer A, spacer B or combination of B and C onto the polyHEMA backbone and the attachment of protein (whether antibody or trypsin) to their active ends.

FIG. 3 (A) Shows recognition of increasing concentrations (1.25-10 μg/ml) of A1AT by rabbit polyclonal anti-A1AT IgG (triangles) or trypsin (circles) coats of polystyrene surface (96 well ELISA plate).

(B) Shows specificity of A1AT detection by surface immobilized trypsin as analyzed by 30 μg/ml of rabbit anti A1AT IgG (circles). Similar concentration of polyclonal rabbit anti-rotavirus IgG served as non-specific control (triangles). In both cases the secondary antibodies was HRP conjugated goat anti rabbit IgG.

FIG. 4: The effect of crosslinking density (expressed in mol % of ethylene glycol dimethacrylate (EGDMA) used for polyHEMA crosslinking) on the film transparency (expressed in optical density) as measured at a wave length of 600 nm, in a dry and hydrated (three different pH values) state.

Shown are the mean values of three experiments±S.D.

FIG. 5: Binding of Alexa Fluor 647 labeled hydrazine to polyHEMA films grafted with three concentrations (0, 3.5 and 7 mg/ml) of activated (GA) LPEI spacers.

Shown are the mean values of three experiments±S.D.

FIG. 6: Binding of Alexa Fluor 488 labeled goat anti-rabbit polyclonal IgG to polyHEMA films grafted with: (A) LPEI spacers of increasing molecular weights (2.5 kDa—white columns, 25 kDa—black columns and 250 kDa—light grey columns) and LPEI-max, 40 kDa—dark grey columns. Note the minimal antibody adsorption to polyHEMA films without spacers; (B) Two concentrations of NHS-^{2K}PEG, four concentrations of NHS-^{3K}PEG-NHS and two concentrations of a mixture of NHS-^{3K}PEG-NHS with NHS-^{2K}PEG.

Shown are the mean values of three experiments±S.D.

FIG. 7: Capturing Alexa 488 labeled OVA by immobilized (via LPEI, 25 kDa of increasing surface densities) polyclonal rabbit anti-OVA IgG on polyHEMA films.

Shown are the mean values of two experiments.

FIG. 8: A1AT recognition (expressed in O.D. arbitrary units) by trypsin (1 mg/ml), immobilized by 7 mg/ml LPEI 25 KDa (open triangles), or 100 mg/ml NHS-^{3K}PEG-NHS (filled squares), or a mixture of 10 mg/ml of NHS-^{3K}PEG-NHS+100 mg/ml of NHS-^{2K}PEG (filled circles) to the polyHEMA film, as analyzed by ELISA. The recognition antibody was rabbit anti-A1AT. The secondary antibody was HRP-conjugated anti-rabbit IgG.

Shown are the mean values of three independent experiments±S.D.

FIG. 9: The reduction in polyHEMA films transparency (expressed in arbitrary O.D. units at 600 nm). (A) after its grafting with LPEI 25 kDa (light grey columns), activating the spacer with GA (dotted columns), linking IgG to the activated end of the spacer (white columns), or linking trypsin to the activated end of the spacer (dark grey columns); (B) after its grafting with NHS-^{3K}PEG-NHS (light grey columns), linking IgG to the activated end of the spacer (white columns).

FIG. 10: Swelling kinetics of polyHEMA films, crosslinked with increasing amounts of EGDMA, as measured by fluid uptake at pH 1.5 (A) and pH 7.5 (B).

Shown are the mean values of three separate experiments±S.D.

FIG. 11: (A) Dry polyHEMA film coatings on top of PillCam domes, before (I, III) and after (II, IV) incubation in SGF (24 h, 37° C., continuous stirring).

Upper panel: 5 mole % of EGDMA; Lower panel: 2.5 mole % of EGDMA.

(B) Weight loss values (% of initial amount) of the polyHEMA film coatings, crosslinked with 2.5, 5, or 7.5% of EGDMA, on top of the PillCam® domes, as measured after 1 h incubation in SGF, followed by 24 or 72 h incubation in SIF.

Shown are the mean values of three separate experiments±S.D.

FIG. 12: The effect of spacer arm density on the glass slide on Alexa Fluor⁵⁵⁵ labeled OVA binding.

Shown are the mean values of two experiments±S.D.

FIG. 13: The effect of different spacers used to immobilize trypsin to the SMSA glass slide on the specific recognition of A1AT as analyzed in SGF by Alexa Fluor647 conjugated anti-rabbit IgG (A), or by HRP conjugated anti-rabbit IgG (B).

Shown are the mean values of two experiments±S.D.

FIG. 14: The effect of the NHS-5kPEG spacer on the non-specific recognition, in vitro, of the A1AT by secondary, Alexa Fluor647 conjugated anti-rabbit IgG.

FIG. 15: Recognizing A1AT in gastric juice by trypsin (100 μg/ml) immobilized to SMSA glass slide with a spacers mixture (10 mM NHS-3 kPEG-NHS+50 mM NHS-2 kPEG) or single spacer (NHS-5kPEG, 30 mM). Detection was performed by Alexa Fluor647 conjugated anti-rabbit IgG, at 635 nm (ex), 660 (em).

Shown are the mean values of two experiments.

FIG. 16: The effect of MAL-5KPEG-NHS spacer density (3-30 mM) on the binding of the Alexa Fluor 555 labeled OVA (grey columns) or OVA-SH (black columns) to the SMSA glass slide.

Shown are the mean values of two experiments.

FIG. 17: The difference in A1AT binding by SH-modified trypsin (100 μg/ml) attached to SMSA glass slide via MAL-^{5k}PEG-NHS or NHS-^{5K}PEG spacers, as analyzed by Alexa Fluor⁶⁴⁷ conjugated anti-rabbit IgG in SGF.

Shown are the mean values of two experiments

FIG. 18: Binding of Alexa Fluor⁵⁶⁸ labeled IgG (two concentrations) to the surface of SMSA-spacer glass slide, previously modified by 30 mM of two types of spacers: NHS-carbonate-NHS or NHS-^{3K}PEG-NHS.

Shown are the mean values of two experiments.

FIG. 19: The effect of spacer (NHS-^{3K}PEG-NHS) density on the binding of the Alexa Fluor⁵⁶⁸ labeled IgG (25 μg/ml) to the SMSA glass slide.

Shown are the mean values of four experiments±S.D.

FIG. 20: SDS-PAGE gel electrophoresis of the non-reduced and reduced TRITC-conjugated (Fab)₂.

Control: intact F(ab)₂; (1): F(ab)₂ obtain after reduction with DTT (Fab:DTT-1:50, final concentration: 0.125 mM); (2) F(ab)₂ obtain after reduction with DTT (Fab:DTT-1:200, final concentration: 0.5 mM); (3) F(ab)₂ obtain after reduction with DTT (Fab:DTT-1:1000, final concentration: 2.5 mM).

Note: the 50 kDa band represents BSA (1.5%) presenting in the sample.

FIG. 21: The binding of increasing concentrations of reduced (Fab:DTT-1:200, final concentration: 0.5 mM) F(ab)₂ fragments and intact (Fab)₂ to the SMSA glass slide pre-treated with 20 mM of MAL-5KPEG-NHS.

FIG. 22: Is a schematic illustration of an in-vivo detecting device used according to one embodiment of the invention

### DETAILED DESCRIPTION OF THE INVENTION

In the following description, various aspects of the present invention will be described. For purposes of explanation, specific configurations, examples and details are set forth in order to provide a thorough understanding of the present invention. However, it will also be apparent to one skilled in the art that the present invention may be practiced without the specific details presented herein and that the examples should not limit the scope of the invention.

Some embodiments of the present invention are directed to a typically swallowable in-vivo device, e.g., a capsule endoscope. Devices according to embodiments of the present invention may be similar to embodiments described in U.S. Pat. No. 7,009,634, entitled “Device And System For In-vivo Imaging”, filed on 8 Mar., 2001, and/or in U.S. Pat. No. 5,604,531 to Iddan et al., entitled “In-vivo Video Camera System”, and/or in International Application number WO 02/054932 entitled “System and Method for Wide Field Imaging of Body Lumens” published on Jul. 18, 2002, all of which are hereby incorporated by reference. An external receiving unit and processor, such as in a work station, such as those described in the above publications could be suitable for use with embodiments of the present invention. Devices and systems as described herein may have other configurations and/or other sets of components. For example, the present invention may be practiced using an endoscope, laparoscope, needle, stent, catheter, etc.

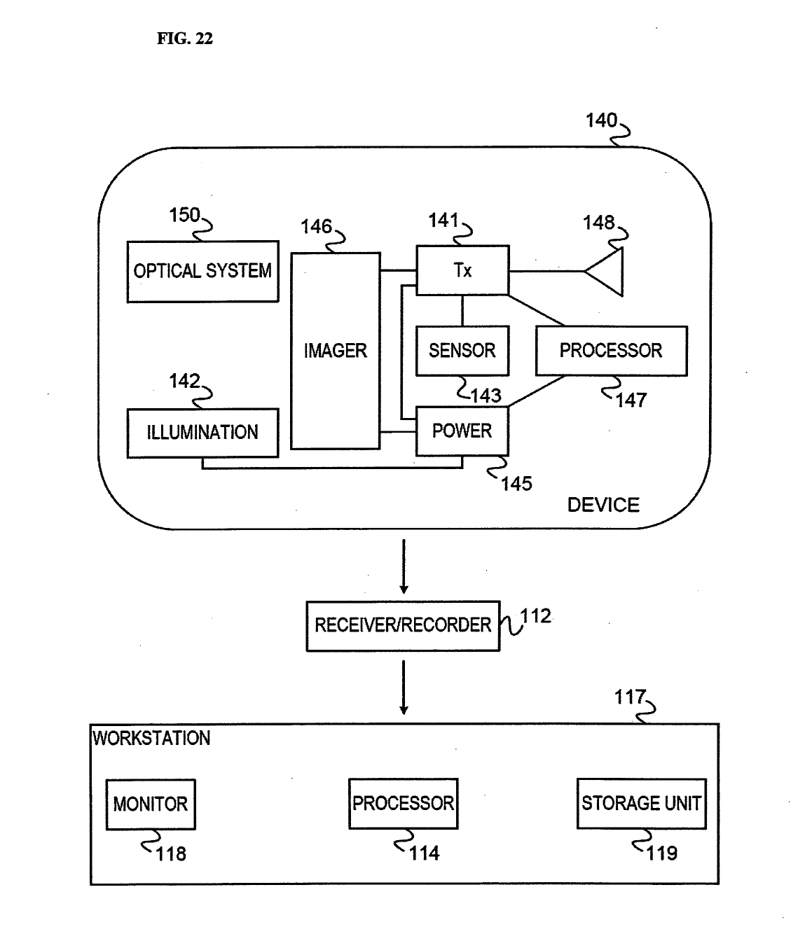

Reference is now made to FIG. 22, which schematically illustrates a system according to an embodiment of the invention. In a preferable embodiment, the system may include a device 140 having a sensor, e.g., light detector or imager 143 equipped with optical filters to match one or more illumination sources 142 to provide fluorescence detection, a power source 145, and a transmitter 141. In some embodiments, device 140 may be implemented using a swallowable capsule, but other sorts of devices or suitable implementations may be used.

Outside a patient's body may be, for example, an external receiver/recorder 112 that include an antenna, a processor, and a display.

Transmitter 141 may operate using radio waves; but in some embodiments, such as those where device 140 is or is included within an endoscope, transmitter 141 may transmit/receive data via, for example, wire, optical fiber and/or other suitable methods. Other known wireless methods of transmission may be used.

Embodiments of device 140 are typically autonomous, and are typically self-contained. For example, device 140 may be a capsule or other unit where all the components are substantially contained within a housing or shell, and where device 140 does not require any external wires or cables to, for example, receive power or transmit information. In some embodiments, device 140 may be autonomous and non-remote-controllable; in another embodiment, device 140 may be partially or entirely remote-controllable.

In some embodiments, device 140 may include in addition to sensor 143 an in-vivo video camera, for example, imager 146 together with optical system 150, which may capture and transmit images of, for example, the gastrointestinal (GI) tract while device 140 passes through the GI lumen. An external receiver/recorder 112 including, or operatively associated with, for example, one or more antennas, or an antenna array, storage unit 119, a processor 114, and a monitor 118. In some embodiments, for example, processor 114, storage unit 119 and/or monitor 118 may be implemented in workstation 117.

Other lumens and/or body cavities may be imaged and/or sensed by device 140. In some embodiments, detector 143 may include, for example, light detector with suitable optical filter adjusted for fluorescence, a Charge Coupled Device (CCD) imager, a Complementary Metal Oxide Semiconductor (CMOS) imager, or other suitable light or image acquisition components.

In some embodiment, transmitter 141 may transmit/receive via antenna 148. Transmitter 141 and/or another unit in device 140, e.g., a controller or processor 147, may include control capability, for example, one or more control modules, processing module, circuitry and/or functionality for controlling device 140, for controlling the operational mode or settings of device 140, and/or for performing control operations or processing operations within device 140.

Power source 145 may include one or more batteries or power cells. For example, power source 145 may include silver oxide batteries, lithium batteries, other suitable electrochemical cells having a high energy density, or the like. Other suitable power sources may be used. For example, power source 145 may receive power or energy from an external power source (e.g., an electromagnetic field generator), which may be used to transmit power or energy to in-vivo device 140.

In some embodiments, power source 145 may be internal to device 140, and/or may not require coupling to an external power source, e.g., to receive power. Power source 145 may provide power to one or more components of device 140 continuously, substantially continuously, or in a non-discrete manner or timing, or in a periodic manner, an intermittent manner, or an otherwise non-continuous manner. In some embodiments, power source 145 may provide power to one or more components of device 140, for example, not necessarily upon-demand, or not necessarily upon a triggering event or an external activation.

Optionally, in some embodiments, transmitter 141 may include a processing unit or processor or controller, for example, to process signals and/or data generated either by imager 146 or sensor 143 or both. In another embodiment, the processing unit may be implemented using a separate component within device 140, e.g., controller or processor 147, or may be implemented as an integral part of imager 146, transmitter 141, or another component, or may not be needed. Preferably the processing is preformed at the receiver and display unit 112 by an appropriate Digital Signal Processor (DSP), In another embodiments the processing unit may include, for example, a Central Processing Unit (CPU), a microprocessor, a controller, a chip, a microchip, a controller, circuitry, an Integrated Circuit (IC), an Application-Specific Integrated Circuit (ASIC), or any other suitable multi-purpose or specific processor, controller, circuitry or circuit. In some embodiments, for example, the processing unit or controller may be embedded in or integrated with transmitter 141, and may be implemented, for example, using an ASIC.

In some embodiments, device 140 may include one or more illumination sources 142, for example one Light Emitting Diode (LED) matching the excitation wavelength of the tagged material and another LED or more, “white LEDs”, or other suitable light sources to illuminate a body lumen or cavity being imaged. An optional optical system 150, including, for example, one or more optical elements, such as one or more lenses or composite lens assemblies, one or more suitable optical filters, or any other suitable optical elements, may optionally be included in device 140 and may aid in focusing reflected light onto imager 146, focusing illuminated light, and/or performing other light processing operations.

In some embodiments, for example, illumination source(s) 142 may illuminate in a pre-defined sequence for example in a periodic manner or an otherwise non-continuous manner to enable both: measuring fluorescence signal by sensor 143 and capturing images by imager 146.

In some embodiments, information sensed by sensor 143 at a certain time period may be displayed on monitor 118 along with the corresponding image information sensed by imager 146 at the same time. In some embodiments, information regarding presence of a specific biomarker in the gastrointestinal system, or information regarding presence of a plurality of different biomarkers that is captured by sensor 143 may be displayed on monitor 118 along side an image of the gastrointestinal system. The image information captured by imager 146 may be captured at the same time that the data sensed by sensor 143 was captured. In other embodiments, information captured by sensor 143 may be displayed onto the corresponding image captured by imager 146 at the same time that sensor 143 sensed the information. For example, an image of the gastrointestinal system may also show information regarding the presence or lack of presence of a biomarker in-vivo. According to some embodiments, an image of an area of the GI tract along with the indication of presence of a biomarker may also provide indication on the in-vivo location of the biomarker. When the image of the area of the gastrointestinal system is acquired at the same time as the optical changes indicating presence of a biomarker are acquired it can be inferred that the biomarker is present in the specific area in the GI tract shown in the combined image.

In some embodiments, the components of device 140 may be enclosed within a housing or shell, e.g., capsule-shaped, oval, or having other suitable shapes. The housing or shell may be substantially transparent or semi-transparent, and/or may include one or more portions, windows or domes which may be substantially transparent or semi-transparent. For example, one or more illumination source(s) 142 within device 140 may illuminate the detection optical window and the excited light detected by sensor 143 while other illumination sources are designed to illuminate a body lumen through a transparent or semi-transparent window or dome that does not contain immobilized recognition factors; and light reflected from the body lumen may enter the device 140, for example, through the same transparent window or dome, or, optionally, through another transparent or semi-transparent portion, window or dome, and may be received by optical system 150 and/or imager 146.

Data processor 114 may analyze the data received via external receiver/recorder 112 from device 140, and may be in communication with storage unit 119, e.g., transferring frame data to and from storage unit 119. Data processor 114 may provide the analyzed data to monitor 118, where a user (e.g., a physician) may view or otherwise use the data. In some embodiments, data processor 114 may be configured for real time processing and/or for post processing to be performed and/or viewed at a later time. In the case that control capability (e.g., delay, timing, etc) is external to device 140, a suitable external device (such as, for example, data processor 114 or external receiver/recorder 112 having a transmitter or transceiver) may transmit one or more control signals to device 140. In another embodiment of the invention, information captured by sensor 143 may be presented with the corresponding image captured by imager 146 at the same time.

Monitor 118 may include, for example, one or more screens, monitors, or suitable display units. Monitor 118, for example, may display in addition to the information captured from sensor 143 and/or transmitted by device 140 also one or more images or a stream of images, e.g., images of the GI tract or of other imaged body lumen or cavity. Additionally monitor 118 may display, for example, location or position data (e.g., data describing or indicating the location or the relative location of device 140), orientation data, and various other suitable data. Other systems and methods of storing and/or displaying collected image data and/or other data may be used.

Typically, device 140 may include few sensors 143 each one configured to detect presence of a different type of biomarker. Instead of or in addition to imager 146 other sensor may be used to, for example, sense, detect, determine and/or measure one or more values of properties or characteristics of the surrounding of device 140. For example, imager 146 may be replaced by a pH sensor, a temperature sensor, an impedance sensor, a pressure sensor, or any other known suitable in-vivo sensor.

According to an embodiment of the invention the in-vivo sensing device is a capsule endoscope. The capsule endoscope typically has a dome shaped optical window at one or both ends of the capsule. Other windows are possible, for example the optical window may be along a side of the device or surrounding the device. Behind the optical window, enclosed within the capsule housing are positioned an image sensor or another light receptor, an optical system for focusing images onto the image sensor and at least one illumination source for illuminating the gastrointestinal (GI) tract through which the capsule endoscope is propagating.

In an embodiment of the invention, the device may be any capsule.

In an embodiment of the invention, there may be provided a device for in-vivo detection of a biomarker in the gastrointestinal tract, the device comprises a housing. The housing may comprise an optical window, and behind the optical window, enclosed within the housing may be positioned a light receptor e.g. imager. In some embodiments, an external surface of the optical window may be coated by a transparent or preferably semitransparent polymer. The polymer may have a recognition factor immobilized thereon via a spacer. According to some embodiments, the imager may be configured to image the optical window. According to an embodiment of the invention, the external surface of the optical window is coated. Typically the optical window is made of a plastic such as Isoplast® or polycarbonate. Other solid phase substrates may be used, for example, glass, silica, or other biocompatible plastics, such as polypropylene and polystyrene.

In some embodiments of the invention, the recognition factor is attached to a recognition platform positioned across the illumination source and light detector/receptor; such platform may be made of various materials, organic or inorganic or a combination of both. The recognition platform may be included in the device by any appropriate manner, such as a coating, enclosing in a compartment, etc. Suitable materials include, but are not limited to, glasses, ceramics, plastics, metals, alloys, carbon, papers, agarose, silica, quartz, cellulose, polyacrylamide, polyamide, and gelatin, as well as other polymer supports, other solid-material supports, or flexible membrane supports. Polymers that may be used as a substrate include, but are not limited to: polystyrene; poly(tetra)fluoroethylene (PTFE); polyvinylidenedifluoride; polycarbonate; polymethylmethacrylate; polyvinylethylene; polyethyleneimine; polyoxymethylene (POM); polyvinylphenol; polylactides; polymethacrylimide (PMI); polyalkenesulfone (PAS); polypropylene; polyethylene; polydimethylsiloxane; polyacrylamide; polyimide; and various block co-polymers. The substrate or support can also comprise a combination of materials, whether water-permeable or not, in multi-layer configurations.

According to an embodiment of the invention, the polymer is polyHEMA. According to a further embodiment, the recognition platform is a polyHEMA film crosslinked with an appropriate amount of ethylene glycol dimethacrylate (EGDMA), e.g., 5 mol %.

In another embodiment of the invention, there is provided a device and a system for in-vivo detection of a cancer biomarker in the gastrointestinal system, the device comprising: a housing comprising an optical window made of glass slide comprising a recognition factor immobilized onto the glass slide via a spacer, behind the optical window, enclosed within the capsule housing are positioned light receptor e.g. imager wherein the imager is configured to image the glass slide. The glass slide according to the embodiment of the invention may be Super Mask™ SuperAmine 2 (SMSA), SuperMask™ SuperAmine, SuperMask™ SuperClean 2, SuperMask™ 16 SuperAldehyde 2, SuperMask™ 16 SuperEpoxy 2 each containing 4, 12, 16, 24, 48, 64, or 192 hydrophobic wells. The Super Mask™ SuperAmine 2 (SMSA) glass slide is characterized by ultra-low intrinsic fluorescence and background noise, containing high density (2×10¹³) of charged amino groups/mm². According to another embodiment, the device includes a chamber in which the glass slide such as SMSA is found, e.g., the glass slide is positioned behind a capsule's dome-shaped optical window, which has the ability to allow gastric fluids flow there through. The free flow of gastric fluids through the capsule typically allows the biological recognition reaction to take place on top of shielded glass surface (i.e. the glass slide). Such a capsule may comprise a dark background which may reduce or eliminate the interference of tissue auto-fluorescence with the fluorescence emitted by the presence of the biomarker; thus increasing signal to noise ratio.

The system in some embodiments of the invention includes the device enclosing the glass slide according to the embodiments of the invention and a transmitter to transmit images from the imager; a receiving system to receive transmitted signals; and a display to display indication of the presence of a marker in-vivo.

In an embodiment of the invention, the biomarker is, for example, without limitation, α1-antitrypsin precursor (A1AT), carcinoembryonic antien (CEA) or CA 19-9. In another embodiment of the invention, it can be any biomarker to gastric cancer or to any other disease that is known or will be known in the art.

According to some embodiments of the invention, the spacer molecules used for immobilizing the recognition factor specific to the biomarker such as a protein, polypeptide, or antibody on the polyHEMA film include (a) linear poly(ethyleneimine) (LPEI); (b) O,O′-bis[2-(N-succinimidyl-succinylamino)ethyl]polyethylene glycol (NHS-^{3K}PEG-NHS); (c) a combination of NHS-^{3K}PEG-NHS and O—[(N-succinimidyl)succinyl-aminoethyl]-O′-methylpolyethylene glycol (NHS-^{2K}PEG); (d) Amino-PEG-Carboxylic acid; (e) Boc-protected-Amino-PEG-Carbonate-NHS; (f) Maleimide-PEG-Carbonate-NHS; (j) Hydroxy-PEG-Aldehyde; (h) Biotin-PEG-Carbonate-NHS.

As can be seen from the Examples section, the a combination of NHS-^{3K}PEG-NHS and O—[(N-succinimidyl)succinyl-aminoethyl]-O′-methylpolyethylene glycol (NHS-^{2K}PEG) was proved as mostly effective in connecting trypsin into the polyHEMA as well as onto the SMSA slide glass.

In an embodiment of the invention, the recognition factor may be a protein, a polypeptide, a polynucleotide or anti-biomarker antibody, or a substrate, that specifically bind to the biomarker.

The terms “specific binding” or “specifically binding” refer to the interaction between a protein, polypeptide, peptide or carbohydrate and a binding molecule, such as a ligand, a substrate, an antibody, a peptide or an aptamer. The interaction is dependent upon the presence of a particular structure (i.e., an antigenic determinant or epitope or substrate binding site in case of an enzyme) of the protein that is recognized by the binding molecule.

Methods of coupling the proteins, polypeptides or anti-biomarker antibodies to the reactive end groups on the surface of the substrate or on the spacer include reactions that form linkage such as thioether bonds, disulfide bonds, amide bonds, carbamate bonds, urea linkages, ester bonds, carbonate bonds, ether bonds, hydrazone linkages, Schiff-base linkages, and noncovalent linkages mediated by, for example, ionic or hydrophobic interactions. The form of reaction will depend, of course, upon the available reactive groups on both the recognition factor/spacer and the antibodies.

In an embodiment of the invention, the trypsin recognition factor may be immobilized onto the recognition platform's surface using any appropriate spacer molecule for capturing the A1AT. According to one embodiment of the invention, the trypsin is anchored on a transparent polyHEMA film, aimed at detecting A1AT on the capsule surface by the use of spacers which are O—[(N-succinimidyl)succinyl-aminoethyl]-O′-methylpolyethylene glycol (NHS-^{2K}PEG) or O,O′-bis[2-(N-succinimidyl-succinylamino)ethyl]polyethylene glycol (NHS-^{3K}PEG-NHS). According to this embodiment, the specific interaction between the immobilized trypsin and A1AT is followed by the addition of primary and labeled or fluorescent secondary antibodies in order to generate a detectable signal. The invention may be also conducted by the use of a labeled primary antibody.

Once the recognition factor attaches to the biomarker found in the gastric fluid, it is necessary to quantify the amount of the biomarker recognized by the recognition factor. This may be done by any appropriate method known in the art.

In an embodiment of the invention, there is provided a method for the in-vivo detection of the presence of a biomarker in the gastrointestinal tract of a subject comprising the steps of: orally administering a device according embodiment provided herein; contacting the orally administered device with a detectable labeled binding agent (e.g. primary antibody) that binds specifically to the biomarker or contacting the orally administered device with a first binding agent (e.g. first antibody) that binds specifically to the biomarker and a second detectable labeled binding agent (e.g. secondary antibody) that binds specifically to the first binding agent; wherein the presence of a bound label as detected by the imager is indicative to the presence of the specific biomarker in the gastrointestinal tract of the subject.

In an embodiment of the invention, the label for the detection of the binding may be a radioisotope, a fluorescent agent, a magnetic bead, gold particles as well as other metal colloidal particles or other appropriate detectable agent or an enzyme label. Fluorescent labels include, for example, Fluorescein, FITC, Indocyanine green (ICG), Coumarin (e.g., Hydroxycoumarin, Aminocoumarin, Methoxycoumarin), R-Phycoerythrin (PE), Fluorescein, FITC, Fluor X, DTAF, Auramine, Alexa (e.g., Alexa Fluor 350, 430, 488, 532, 546, 555, 568, 594, 633, 647, 660, 680, 700, 750), BODIPY-FL, Sulforhodamine, Carbocyanine (e.g., Cy2, Cy3, Cy3.5, Cy5, Cy5.5, Cy7), Rhodamine, XRITC, TRITC, Lissamine Rhodamine B, Peridinin Chlorphyll Protein (PerCP), Allophycocyanin (APC), PE-Cy5 conjugates (e.g., Cychrome, Tri-Color, Quantum Red.), PE-Cy5.5 conjugates, PE-Cy7 conjugates, PE-Texas Red conjugates (e.g., Red613), PC5-PE-Cy5 conjugates, PerCP-Cy5.5 conjugates (e.g., TruRed), APC-Cy5.5 conjugates, APC-Cy7 conjugates, ECD-PE-Texas Red conjugates, Sulfonated Pyrene (e.g., Cascade Blue), AMCA Blue, and Lucifer Yellow.

Isotope labels include ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re. Enzyme labels include peroxidase, beta-glucuronidase, beta-D-glucosidase, beta-D-galactosidase, urease, glucose oxidase plus peroxidase, and alkaline phosphatase. Enzymes can be conjugated by reaction with bridging molecules such as carbodiimides, diisocyanates, glutaraldehyde, and the like. Enzyme labels can be detected visually, or measured by calorimetric, spectrophotometric, fluorospectrophotometric, amperometric, or gasometric techniques. Other labeling systems, such as avidin/biotin, colloidal gold (e.g., NANOGOLD), Tyramide Signal Amplification (TSA), are known in the art, and are commercially available (see, e.g., ABC kit, Vector Laboratories, Inc., Burlingame, Calif.; NEN Life Science Products, Inc., Boston, Mass.; Nanoprobes, Inc., 95 Horse Block Road, Yaphank, N.Y.). The use of any of those labels is subjected to toxicology tests and the approval of the health authorities.

According to some embodiments of this invention, the measurement of the fluorescent signal may be performed by any appropriate means, including a miniature spectrophotometer or a photomultiplier or a narrow band illumination source and a photodetector covered by high pass- or a notch filter blocking the excitation light and detecting only the emission light capable of recording the fluorescent signal produced by the reaction between the captured biomarker and the fluorescent preliminary or secondary antibody.

In the case that the binding agent binds to the complex on the optical window, the colored binding agent will be in the field of view of the image sensor and may appear as a colored spot or other shaped mark in an image being obtained by the image sensor also termed here as “imager”.

The presence of the labeled binding agent which is typically an antibody may be detected, either by being viewed and imaged by the image sensor of the capsule endoscope or by other suitable detecting means which may be included in the capsule endoscope, for example, other optical detectors or a radiation detector.

Data sensed by the in-vivo device according to embodiments of the invention, are transmitted in an embodiment of the invention to an external receiver and are viewed and/or analyzed by a processor outside the body. Data sensed by the device, for example, image data, may include indication of the presence of the biomarker. The presence of the binding agent may be indicative of the presence of the marker in the lumen being examined and as such may indicate to a physician that the patient being examined may be in danger of developing cancer or other pathologies.

According to an embodiment of this invention, the reaction from the step of exposing the coated device or the device enclosing the glass slide to a primary antibody and measuring the detection lasts no more than 30 minutes. According to an embodiment of this invention, the reaction lasts no more than 20 minutes. According to an embodiment of this invention, the reaction lasts no more than 15 minutes.

While performing in vitro experiments it is possible to quantify the amounts of A1AT, recognized by the immobilized trypsin, by reacting the entrapped biomarker with anti-A1AT antibody followed by recognition of HRP or fluorescently labeled secondary antibody such as anti-rabbit IgG. However, this approach might not be applicable for in-vivo condition diagnosis; therefore an alternative detecting strategy is required to generate an intensive photonic signal that can be detected by the video capsule. According to an embodiment of the invention, fluorescently labeled polystyrene beads (FluoSpheres® beads, containing surface-pendent carboxylic moieties, making them suitable for covalent coupling of proteins and other amine-containing biomolecules) may be used in order to detect fluorescent signals from wells in the SMSA glass slide, onto which FluoSpheres® beads were attached specifically.

Other types of “nanocontainers” that can be used, e.g. iron oxide, gold particles and polysaccharide based Nanoparticles.

In accordance with some embodiments of the invention, there is provided a kit comprising: a device according to the embodiment of the invention and a binding agent capable of specifically binding a biomarker which is labeled by a detectable label or a combination of a first binding agent that binds specifically to a biomarker and a second detectable labeled binding agent that binds specifically to the first binding agent; wherein the binding agent or the combination are either contained in a separate containers or are included in the device. In an embodiment of the invention, if the binding agent or the combination of a first and a second binding agent are in separate containers, a leaflet is attached explaining the instructions for oral administration thereof.

According to other embodiments the binding agent may be in any other suitable form, such as in a powder, spray or suspension or in a tablet.

In some embodiments of the invention, there is provided a transparent film coated by a recognition platform that binds to the specific biomarker; wherein the recognition platform comprises a polymer and a recognition factor conjugated by a spacer. In an embodiment of the invention, the transparent film may be coating any capsule.

In some embodiments of the invention, there is provided a polycarbonate film coated by a recognition platform that binds to the specific biomarker; wherein the recognition platform comprises a polymer and a recognition factor conjugated by a spacer. In an embodiment of the invention the polycarbonate film may be coating any capsule.

In an embodiment of the invention, there is provided a glass slide that binds to a specific cancer biomarker; wherein the glass slide comprises a recognition factor immobilized thereon via a spacer.

In an embodiment of the invention, the glass slide may be enclosed in any capsule.

According to one embodiment, the in-vivo device may include a sensor such as a sensor of electrical charge to sense a change in electrical charge which may indicate a change in the configuration of the recognition factor due to its interaction with the biomarker.

Administering a device in-vivo may be done in any suitable way such as by swallowing (by the patient) or otherwise inserting the device into the patient's GI tract by attaching it to an endoscope or any other suitable in-vivo device.

The timing of the different administrations may be planned such to allow sufficient time for the recognition factor to bind the biomarker and only then for the biomarker-recognition factor complex to bind the tagged binding agent.

The invention also relates in one of its embodiment to a device, system and method for detection the biomarker, in which two or more different recognition factors are attached to the recognition platform. According to this embodiment, the detection of two biomarkers more is possible by administering one device. The recognition factors may be attached to the coating onto the optical window or elsewhere in the capsule or to a solid support enclosed in the device or to any combination thereof. According to this embodiment, the labeled primary antibody or the secondary antibody may be detected by one or more of the methods provided herein.

According to one embodiment an acid reducing agent may be administered to the patient. Acid reducing agents, such as known antacids (e.g., Maalox, Rolaids etc.) will typically raise and buffer the pH level in the stomach, thus providing a more stable environment for the recognition factor and the binding agents (typically proteins) and for the markers themselves. For example, acid reducing agents may neutralize pepsin in the stomach and, at least in part, may inhibit the activation of protease precursors that are secreted from the pancreas into the bowel, thus providing an environment essentially free of active pepsin for the procedure of the invention. According to one embodiment a pH level of between about 6.0 to about 7.4 may be desirable. According to one embodiment pH in the range of 6-8 is optimal for stable trypsin (as well as other relevant proteases that can bind A1AT)/A1AT complex formation. However, other pH levels may also be obtained according to embodiments of the present invention. For example, according to one embodiment a pH of above 5.5 may be obtained.

The kits and the method for the detection of the biomarker may be used for the detection of the success of a treatment to the gastric cancer, wherein a reduced or eliminated biomarker concentration is indicative to a successful treatment.

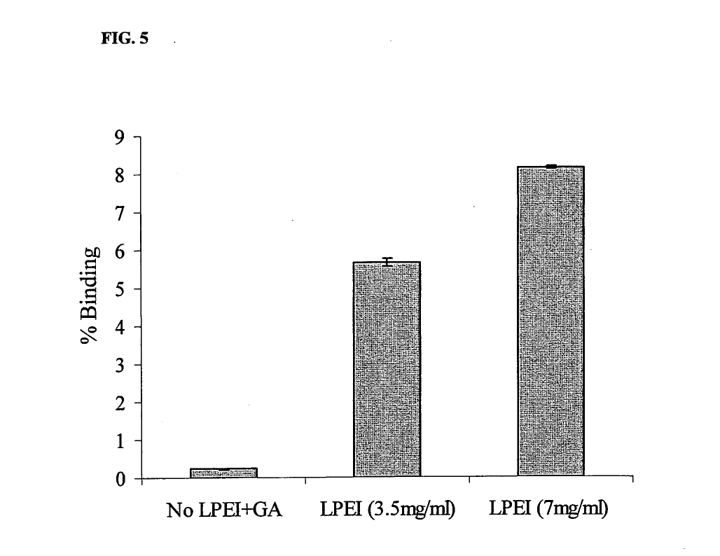

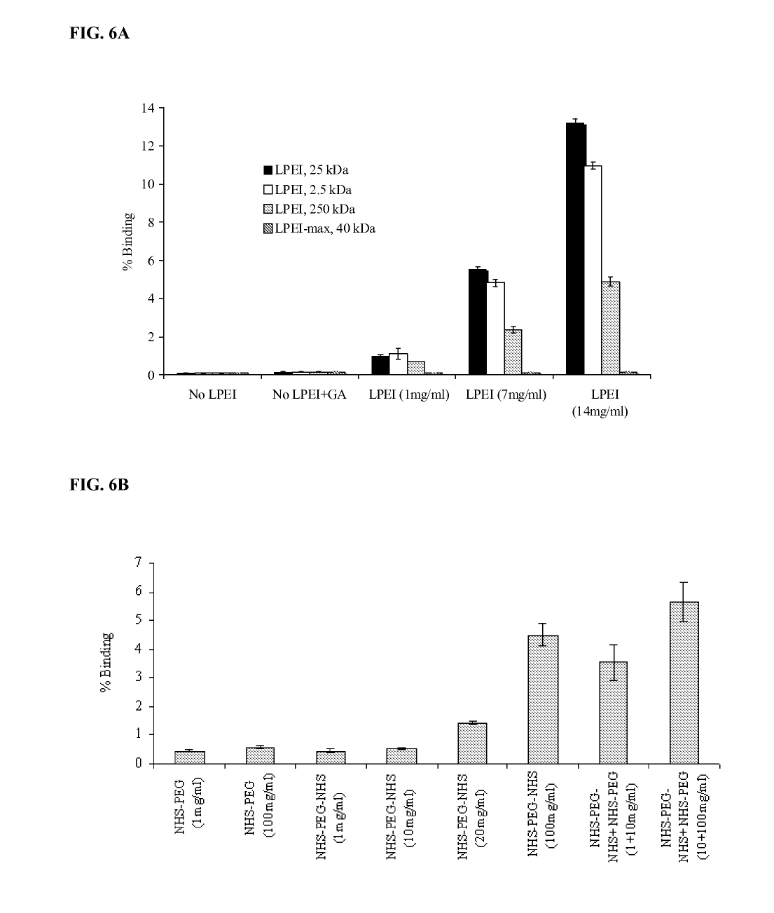

Reference is now made to FIGS. 5 and 6, which show that the presence of the spacer arms was important for binding significant amounts of the fluorescent molecule, which do not bind well to a polyHEMA film that did not contain a spacer. FIG. 6 further shows that the LPEI spacer was more effective that the NHS-3 KPEG-NHS or its mixture with NHS-2 KPEG in binding the goat anti-rabbit polyclonal IgG to the polyHEMA films.

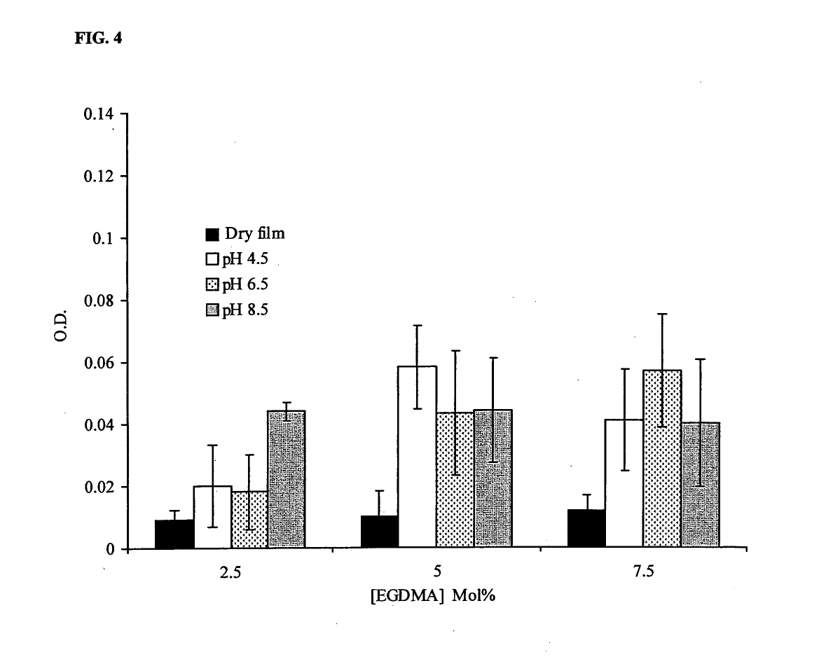

Reference is now made to FIG. 4A, which demonstrates that out of all LPEI activated spacers, the 25 kDa was the best in biding the protein compared with non-grafted polyHEMA films, although pretreated with gluteraldehyde.

With reference to FIG. 8, it is demonstrated that when the NHS-PEG-NHS spacer was used to link trypsin to the surface of the polyHEMA, A1AT was captured significantly better compared with the binding accomplished when LPEI was used as a spacer arm. Overall, the use of a spacer for trypsin immobilization was crucial for A1AT capturing. The inhibitor was hardly recognized when trypsin was conjugated directly to the surface of the polyHEMA film.

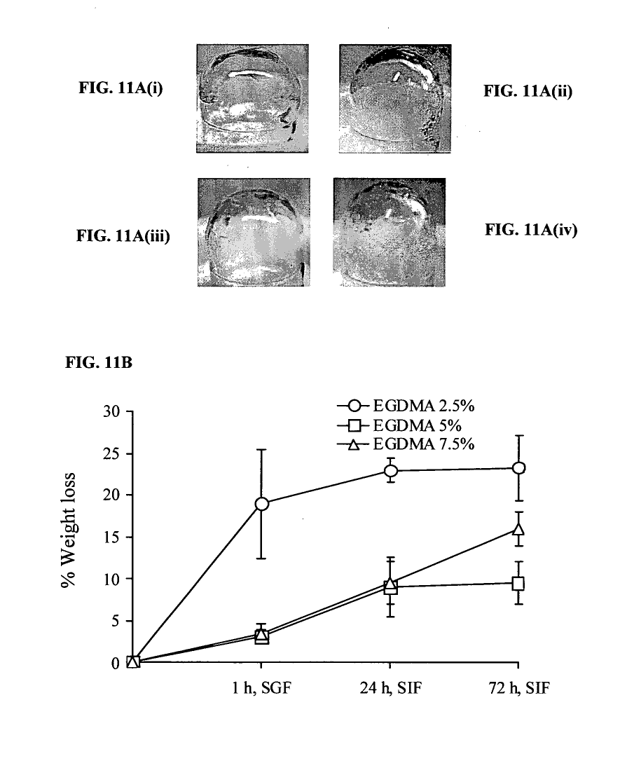

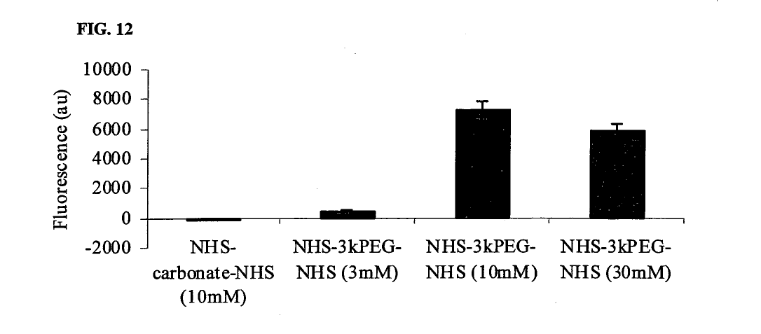

Reference is now made to FIG. 12, relating to the use of spacer with an SMSA glass slide, and showing that the NHS-carbonate-NHS spacer was non-efficient in the specific binding of the ovalbumin, apparently due to its high non-specific adsorption to the naked glass surface. On the other hand, concentration-dependent binding of the ovalbumin (OVA) was observed when the surface of the glass slide was modified with increasing amounts of the NHS-^{3K}PEG-NHS spacer.

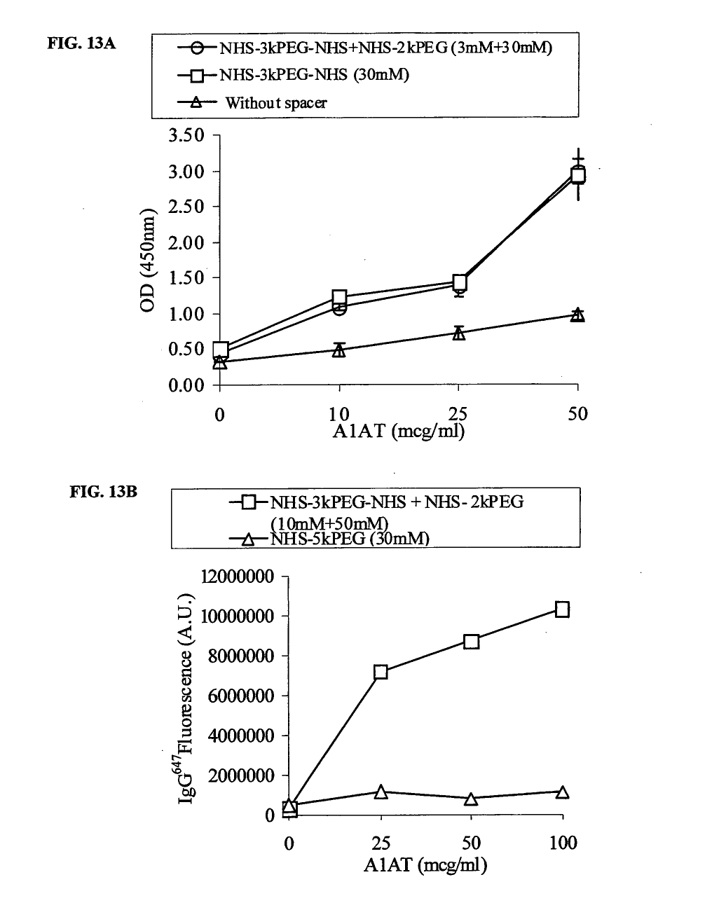

In reference to FIG. 13, it is demonstrated that the fluorescent signal was much higher when a mixture of NHS-^{3K}PEG-NHS and NHS-^{2K}PEG was used, compared with the use of NHS-^{2K}PEG only when recognizing A1AT by trypsin attached to an SMSA glass slide via various spacer arms. FIG. 13B shows that the use of a spacer was crucial for the detection process.

Various aspects of the invention are described in greater detail in the following Examples, which represent embodiments of this invention, and are by no means to be interpreted as limiting the scope of this invention.

### EXAMPLES

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

Relating to all of the examples below, linear polyethyleneimine (LPEI) and polyethyleneimine “MAX” (LPEI-Max) were purchased from Polyscience, Warrington, Pa., USA. Alexa-Fluor 488 labeled goat anti-rabbit IgG (H+L) and Alexa-Fluor 488 labeled ovalbumin (OVA) were purchased from Molecular Probes, Eugene, Oreg., USA. Rabbit anti-OVA polyclonal IgG, 2-hydroxyethyl methacrylate (HEMA), ethylene glycol dimethacrylate (EGDMA); hexamethylenediamine, glutaraldehyde (GA) grade I, benzoyl peroxide, trypsin from bovine pancreas (≧10,000 BAEE units/mg protein), O,O′-bis[2-(N-succinimidyl-succinylamino)ethyl]polyethylene glycol 3,000 (NHS-PEG-NHS), O—[(N-succinimidyl)succinyl-aminoethyl]-O′-methylpolyethylene glycol 2,000 (NHS-PEG), α1-antitrypsin (A1AT), anti rabbit horseradish peroxidase (HRP)-conjugated IgG, protein-A conjugated to HRP and 3,3′,5,5′-tetramethylbenzidine (TMB) reagents and Rabbit polyclonal anti A1AT IgG were all purchased from Sigma, St Louis, Mo., USA. Rabbit anti rotavirus IgG were obtained from Novamed, Jerusalem, Israel. All solvents were analytical grade. Water was purified by reverse osmosis.

### Example 1

### Preliminary A1AT Sandwich ELISA Analysis

ELISA 96 wells plates were coated with 10 μg/ml of rabbit polyclonal anti A1AT IgG (phosphate buffer solution (PBS) pH 7.4, 37° C., 45 minutes) and other ELISA 96 well were coated with 10 μg/ml of trypsin (PBS, pH 5, 37° C., 45 minutes). After a PBS rinse, each plate was washed twice with PBS supplemented with Tween-20 (0.05%). Non specific binding was blocked with 10% w/v (bovine serum albumin) BSA in PBS (37° C., 45 minutes). After the addition of increasing concentrations (0-10 μg/ml) of A1AT (50 μl/well) the plate was incubated (37° C., 45 minutes) and subsequently rinsed with PBS. The detecting antibody, rabbit polyclonal anti human A1AT, (10 μg/ml in blocking solution) was added to each well, followed by a similar incubation and PBS rinse. In the case of surface immobilized trypsin, specificity of A1AT detection was assessed by 30 μg/ml of rabbit anti A1AT IgG. Similar concentration of polyclonal rabbit anti-rotavirus IgG served as non-specific control. In both cases the secondary antibodies was goat anti rabbit-HRP or protein-A-HRP conjugates (1:5000 in PBS). After rinsing, the wells were reacted with a TMB reagent (100 μl/well). The color reaction was stopped with H₂SO₄ 1M. Color intensity was measured in a microplate reader at 450 nm.

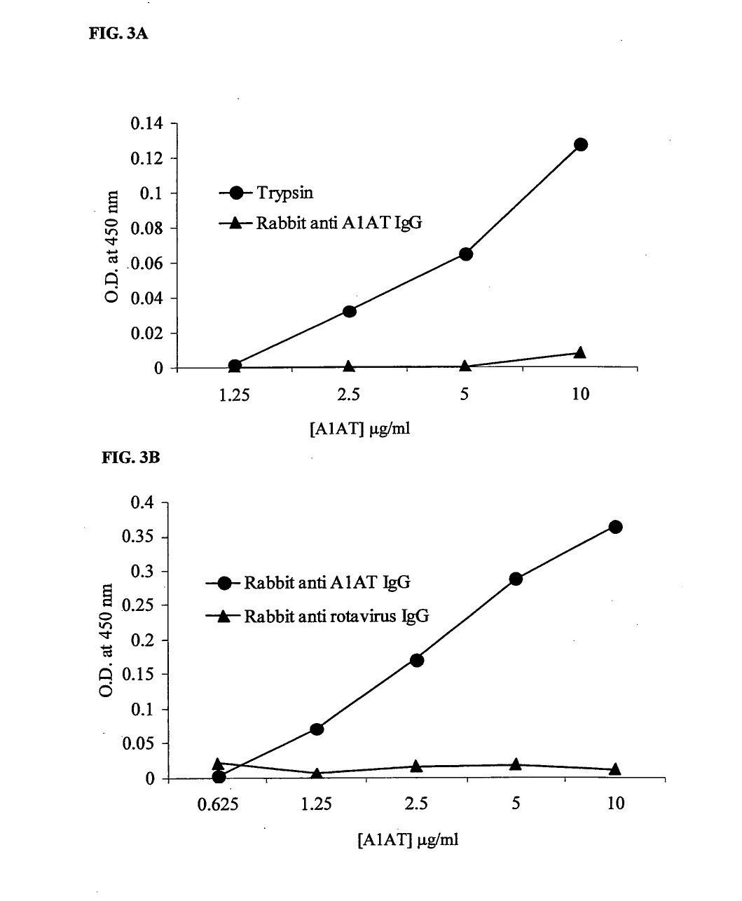

The results, as shown in FIG. 3, teach that the rabbit polyclonal anti A1AT IgG coat was unable to capture A1AT. However, trypsin, with its high specificity to A1AT was able to exert a recognition reaction.

### Example 2

### HEMA Polymerization and Films Preparation

In light of the findings of Example 1, a polymeric platform into which trypsin could be immobilized while, at the same time, maintaining its recognition capabilities was prepared.

10 mM of HEMA was polymerized in the presence of 2.5, 5 or 7.5 mole % of EGDMA (single step polymerization and crosslinking) in 50 ml of acetonitrile, by shaking at 80° C. for 24 h, under dry nitrogen, using 0.5 mole % of benzoyl peroxide as an initiator. The polymerized HEMA, crosslinked with EGDMA (related to herein as polyHEMA), solution was then cooled down to room temperature. Acetonitrile was evaporated and monomer residues were extracted and removed with diethyl ether. The obtained soft mass was dissolved in absolute methanol and cast onto 55 mm (internal diameter) flat Teflon molds and over the polycarbonate surface of the Pillcam® capsules (Given Imaging, Yokneam, Israel). The molds were dried at room temperature (24 h) to obtain transparent films, 0.2-0.5 mm thick (measured by Mitutoyo micrometer, Aurora, Ill., USA), depending on the concentration of EGDMA used. The larger the concentration of EGDMA, the thinner the film obtained. A decision on the optimal EGDMA concentration was taken after comparing the physical properties of the three types of films, originating from the three different concentrations of EGDMA tested.

### Physical Characterization of the PolyHEMA Films

Swelling properties of the polyHEMA films were measured in simulated gastric fluid USP, pH 1.5, without pepsin (SGF) and in 35 mM NaCl aqueous solutions of either pH 5.5 or pH 7.5 by immersing, separately, pre-weighed dry films, for 1, 4 or 24 h under gentle shaking at 25° C. The weight gain of the hydrated (gently blotted dry) films was measured and expressed as the fraction (%) increase from the initial dry weight. The swelling properties of all films were similar, i.e., 33±2% within 1 h. The weight change of the hydrated films (ΔW) from the initial weight (Wi) of the polyHEMA was calculated according to the following equation:

$$\% weight change = \frac{Δ W}{{W}_{i}} \times 100 \qquad Equation 1$$

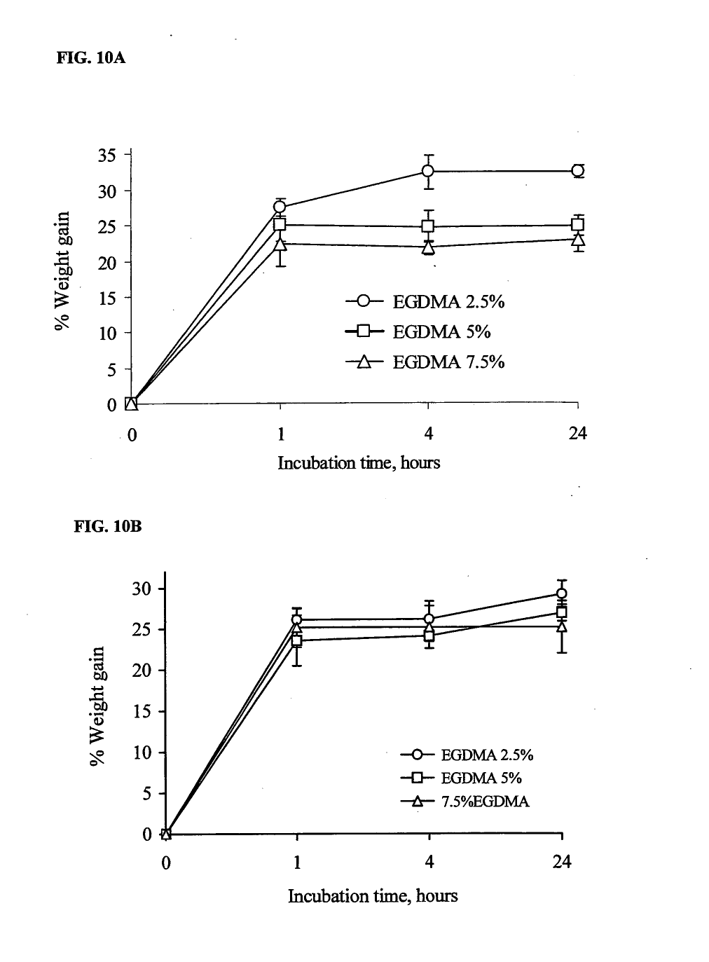

The typical fluid uptake of the polyHEMA films crosslinked with increasing amounts of EGDMA, at pH 1.5 and pH 7.5 is shown in FIG. 10. At both buffers swelling continued for 1 hour and reached a maximum of 32.5% weight gain, excluding the polymer which was crosslinked with the lowest amount of EGDMA (2.5 mole %), at pH 1.5, in which case, fluid uptake was similar after 24 hours. Non-crosslinked polyHEMA (no addition of EGDMA) dissolved completely within 1 h (data not shown).

Tensile strength and adhesion to the polycarbonate surfaces were determined by a TA.XT Plus texture analyzer (Stable Micro Systems, Surrey, UK). Po1yHEMA films (2×2 cm, n=4), either in dry or hydrated (SGF, 2 hours) states, were stretched at a speed of 0.5 mm/sec and a trigger force of 2 gF. Force vs. time plots were drawn and the modulus of elasticity was calculated by the apparatus data acquisition program. The same apparatus was used to assess the detachment force required to separate the films from the surface of the polycarbonate capsule (four repeated tests for each film specimen). In this set of studies a 5 kg-load cell in its compression mode was used, the compression speed was 1 mm/sec and the trigger force was 5 gF (results shown in Table I). Table I shows the mean values of four measurements at each given concentration of EGDMA, relating also to the standard deviation.

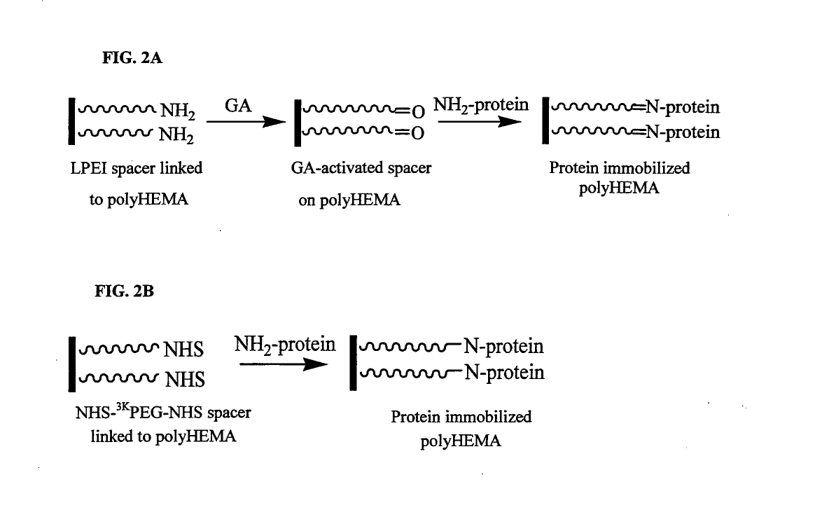

Despite the similar swelling properties of the films, as detailed above, the film prepared with 5.0 mole % EGDMA adhered better to the polycarbonate capsule upon polymer hydration after incubation in SGF (no pepsin) for 24 h (Table I). Photographs of stable and unstable polyHEMA coatings, taken at the end of the stability test are shown in FIG. 2. Based on the findings of weight loss of the films that were coated on top of the polycarbonate dome and incubated with SIF (Table II), the films mechanical (Table I) and swelling (FIG. 10), the polymer crosslinked with 5 mol % EGDMA was selected for the continuation of the study.

The effect of crosslinking density on the transparency of the polyHEMA film in dry and hydrated states, as measured at three pH values, is shown in FIG. 4. The dry films were transparent (typical average O.D. value of 0.01), while wetting the films reduced insignificantly their transparency in a pH independent manner, a finding which may indicate that within 1 h in gastric fluids, the films transparency may be reduced 5-fold (FIG. 10).

TABLE I
The effect of EGDMA amount on the modulus of elasticity,
adherence to polycarbonate capsule and the stability of the
film coat on top of the polycarbonate capsules.

| | | Adhesion | Adhesion | Film stability |
|---|---|---|---|---|
| | Modulus of | force onto | force onto | on poly- |
| EGDMA | elasticity | polycarbonate | polycarbonate | carbonate |
| (Mole %) | (N sec⁻¹) | (dry) (gF) | (hydrated) (gF) | (% wt loss)¹ |
| 2.5 | 0.02 ± 0.004 | 0.7 ± 0.03 | 90 ± 20 | 21.2 ± 6.2 |
| 5.0 | 0.29 ± 0.035 | 0.3 ± 0.09 | 46 ± 7 | 9.6 ± 0.7 |
| 7.5 | 0.22 ± 0.02 | 0.2 ± 0.08 | 16 ± 4 | 12.1 ± 3.0 |

¹As determined gravimetrically (Equation 1), after continuous stirring of coated polycarbonate dome (24 h, 37° C.) in SGF (no pepsin). Shown are the mean values of 4 separate measurements ± S.D.

Physical stability (integrity) of polyHEMA film coats on the polycarbonate capsules was measured visually and gravimetrically after incubation (37° C.) in SGF for 1 h or 24 h, or incubation (37° C.) in simulated intestinal fluid USP, pH 6.8, no pancreatin (SIF) for 24 h (results shown in Table II). Table II shows the mean values of four measurements at each given concentration of EGDMA, relating also to the standard deviation.

TABLE II
Physical stability (determined gravimetrically) of the polyHEMA films
on polycarbonate surface after incubation in SGF (1 or 24 h) and
in SIF (24 h).

| | | |
|---|---|---|
| EGDMA | % wt loss, SGF | % wt loss, SIF |

| (Mole %) | 1 h | 24 h | 24 h |
|---|---|---|---|
| 2.5 | 19 ± 6.5 | 21.2 ± 6.2 | 23 ± 1.4 |
| 5 | 3.1 ± 0.9 | 9.6 ± 0.7 | 9 ± 3.6 |
| 7.5 | 3.4 ± 1.2 | 12.1 ± 3.0 | 9.5 ± 2.5 |

As shown in FIG. 4, which summarizes the effect of EGDMA on the transparency of the polyHEMA films in dry and hydrated states, at three pH values, crosslinking had negligible effect on light transmittance through the films. The overall OD values did not exceed 0.06, with a pH independent increase, resulted by wetting the films.

Based on the above characterization studies the polyHEMA polymer crosslinked with 5 mole % of EGDMA was selected to serve as the immobilization platform.

### Example 3

### Spacer Selection for Immobilizing Trypsin to the PolyHEMA Films

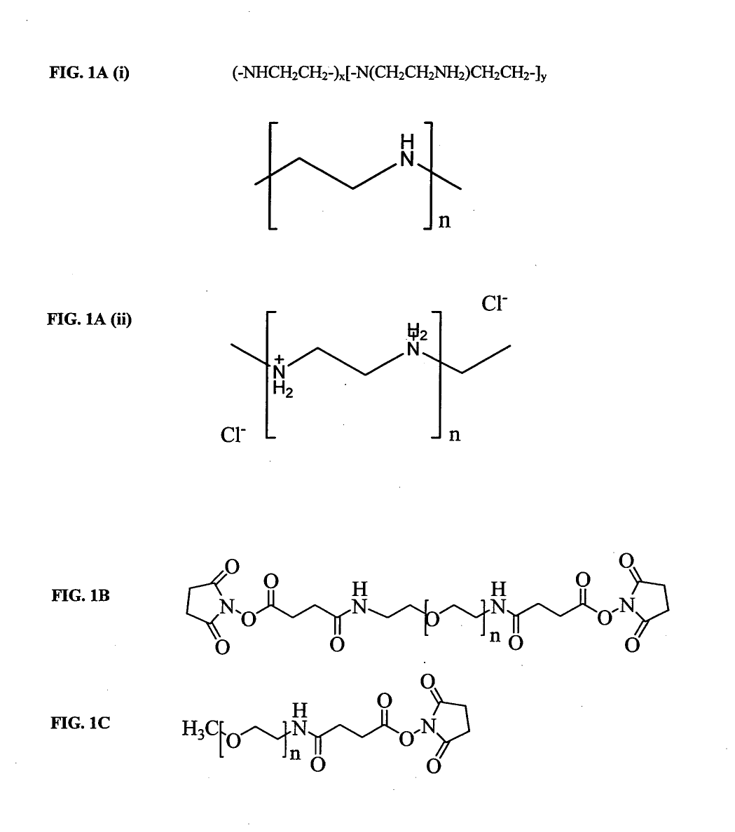

In the course of the study it became apparent that the use of a spacer arm to immobilize the capture moiety is unavoidable. The next set of studies was geared at employing trypsin for capturing A1AT. The following spacers were tested for trypsin immobilization to the polyHEMA film: (a) linear polyethyleneimine (LPEI) of increasing molecular weights (2.5, 25 and 250 kDa); (b) polyethyleneimine “MAX” hydrochloride salt (LPEI-Max; M_{W} 40 kDa); (c) PEG-based spacer: the homobifunctional O,O′-bis[2-(N-succinimidyl-succinylamino)ethyl]polyethylene glycol 3,000 (NHS-^{3K}PEG-NHS), or a mixture of NHS-^{3K}PEG-NHS with the monofunctional O—[(N-succinimidyl)succinyl-aminoethyl]-O′-methylpolyethylene glycol 2,000 (^{2K}PEG-NHS). The chemical structures of these spacers are shown in FIG. 1.

### The LPEI Spacers

The LPEI spacers (see FIG. 1) (both LPEI and LPEI-max) were attached to the polyHEMA films (see FIG. 2) by immersing (gentle shaking, 55° C., 16 h), separately, the dry films with increasing concentrations (3.5, 7 or 14 mg/ml) of the various spacers, followed by a water rinse to remove non-reacted residues. Spacer attachment to the polyHEMA films was verified by elemental analysis (Table III), which revealed that, depending on the LPEI type, total nitrogen content was in the range of 0.39-0.58%, corresponding to 4.33-6.44% of total LPEI bound to the polyHEMA film.

TABLE III
Microanalysis of the LPEI-treated polyHEMA films

| | | | | LPEI, |
|---|---|---|---|---|
| | | LPEI, 2.5 kDa- | LPEI, 25 kDa- | 250 kDa- |
| | polyHEMA | polyHEMA | polyHEMA | polyHEMA |
| Atom | Mass % | Mass % | Mass % | Mass % |
| C | 71.72 | 59.17 | 57.31 | 55.25 |
| H | 7.81 | 8.04 | 7.91 | 7.96 |
| N | 0 | 0.46 | 0.58 | 0.39 |
| % of | — | 5.1 | 6.44 | 4.33 |
| bound | | | | |
| LPEI | | | | |
| (from | | | | |
| total) | | | | |

### Activation of the Grafted LPEI Spacers

The use of LPEI spacers required activation by gluteraldehyde (GA) of the grafted molecules, to enable the formation of an imine bond between the spacer and the protein's primary amine (see FIG. 2). The end amine groups of the grafted LPEI or the LPEI-Max spacers were activated by bathing (room temperature, 45 minutes) the modified films in 1% w/v GA in water with a subsequent water rinse. Activation was verified by incubating (37° C., 2 h) the films (4 mg) with 100 μL of 5 μg/mL of Alexa Fluor 647 labeled hydrazine, followed by a water rinse. The reaction with the activated spacers was verified by measuring fluorescence intensity (excitation: 648 nm, emission: 668 nm) in a microplate reader (Synergy HT Multi Mode, Bio-Tek, VT, USA), using GA-treated polyHEMA (no spacer grafts) as controls.

FIG. 5 shows that the fluorescent hydrazine reacted with the activated polyHEMA films in a spacer-density dependent manner and that the presence of activated LPEI grafts was important for binding significant amounts of the fluorescent molecule (5.5- and 8-fold binding of the films containing 3.5 and 7 mg/ml of LPEI respectively, compared with polyHEMA control film which was treated with GA but did not contain a spacer.

### Protein Binding Capacity of the Activated Films

The ability of the activated polyHEMA-LPEI films to interact with a proteinacious probe was tested, preliminarily, towards fluorescently labeled goat anti-rabbit polyclonal (H+L) IgG.

The activated films (4 mg specimens) were incubated (4° C., overnight) with 50 μL of 50 μg/ml of Alexa Fluor 488 labeled goat anti-rabbit polyclonal (H+L) IgG, in PBS pH 7.4. Unbound antibody was removed by washing with PBS containing 0.1% w/v Tween-20 and the existence of bound fluorescent IgG was verified spectrofluorimetrically (excitation: 485 nm, emission: 525 nm) in a microplate reader, using GA-treated polyHEMA (no spacer grafts) as controls.

To assess the relationship between the surface density of the immobilized recognition protein and the capability of the polyHEMA platform to capture a biomarker, polyclonal rabbit anti-OVA IgG was grafted onto the polyHEMA film using increasing concentrations (elevated surface density) of the LPEI 25 kDa spacer. Thus, polyclonal rabbit anti-OVA was conjugated to the activated polyHEMA-LPEI film by its incubation (PBS pH 7.4, 4° C., overnight) with 15 μg/ml of the antibody, followed by a Tween-20 (0.1% w/v in PBS) rinse to remove unbound residues and 1% w/v dry milk in PBS (room temperature, 2 h) to block nonspecific protein binding. The films were then incubated (PBS, pH 7.4, 45 minutes) with 2 μg/ml of Alexa 488 labeled OVA. Unbound antigen was removed by a Tween-20 (0.1% w/v in PBS) rinse.

In this experiment the binding capacity of the polyHEMA film grafted, separately, with all four LPEI spacers, with increasing (1, 3.5, 7 and 14 mg/ml) spacer densities, was compared. The study was conducted in a 96 well plate and fluorescence (excitation: 485 nm, emission: 525) was monitored in the microplate reader.

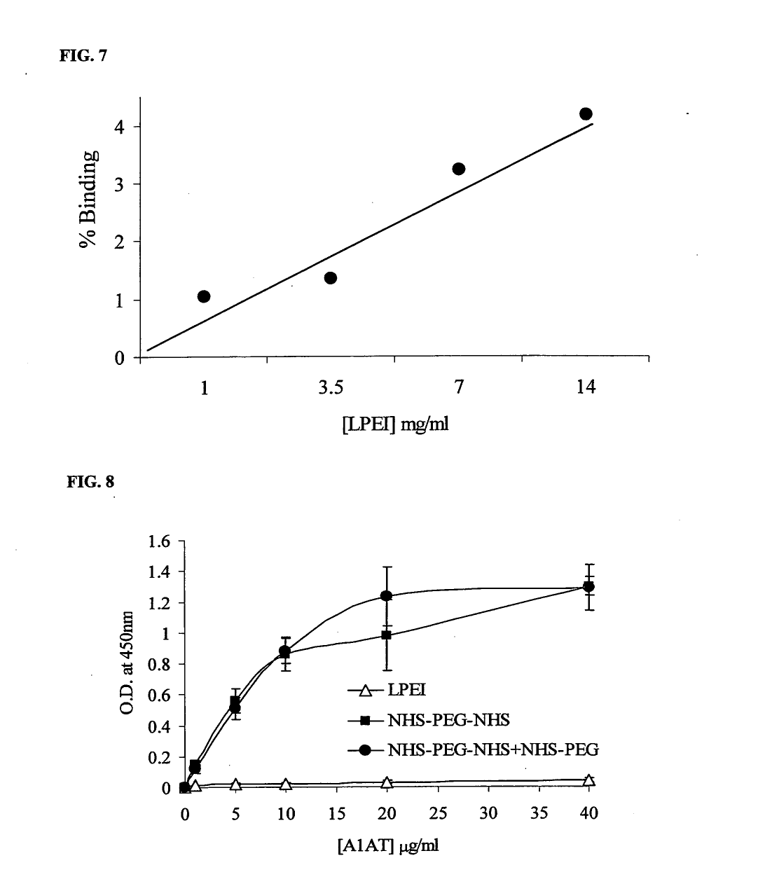

The ability of the grafted polymer to identify, in turn, a model antigen, Alexa 488 labeled OVA, is shown in FIG. 7. Despite a relative low capturing capacity of this specific system (binding in the order of magnitude of 4% of initial amount of OVA), a linear relationship between the surface density of the grafted antibody and the overall recognition capacity of the polymer was demonstrated.

Non-specific binding of Alexa 488 labeled OVA to polyHEMA films that were previously incubated with polyclonal rabbit anti-OVA IgG without the LPEI spacer was also measured and found to be negligible (data not shown).

### The PEG-Based Spacers

In separate studies increasing concentrations (10-100 mg/ml) of NHS-^{3K}PEG-NHS, or a mixture of NHS-^{3K}PEG-NHS with NHS-^{2K}PEG in PBS (pH 7.4), were immersed (gentle shaking, 3 h, 25° C.) with the dry polyHEMA films followed by a water rinse to remove non-reacted residues. See FIG. 1 for the structures of the PEG spacers and FIG. 2 for the grafting to the polyHEMA.

### Protein Binding Capacity of the Modified Films

The ability of the PEG-based spacer-containing polyHEMA films to interact with proteinacious probe was tested towards fluorescently labeled goat anti-rabbit polyclonal (H+L) IgG as described above. In separate studies, films containing increasing concentrations (1, 10, 20 or 100 mg/ml) of NHS-^{3KP}EG-NHS, or films containing mixtures of NHS-^{3KP}PEG-NHS and NHS-^{2K}PEG spacers (1+10 or 10+100 mg/ml, respectively) were incubated (25° C., 2 h) with 50 μg/mL of Alexa Fluor 488 labeled goat anti-rabbit polyclonal (H+L) IgG, in PBS pH 7.4. Unbound antibody was removed by a Tween-20 (0.1% w/v in PBS) rinse. The existence of bound fluorescent IgG was verified spectrofluorimetrically (excitation: 485 nm, emission: 525 nm) in a microplate reader, using NHS-^{2K}PEG-coated film as control.

As shown in FIG. 6, in general, LPEI was more effective than the NHS-3 KPEG-NHS or its mixture with NHS-2 KPEG in binding the goat anti-rabbit polyclonal IgG to the polyHEMA films. FIG. 4A demonstrates that out of all LPEI activated spacers, the 25 kDa was the best in biding the protein (13-, 11- or, 5-fold for 25 kD, 2.5 kD and 250 kD, respectively), compared with non-grafted polyHEMA films, although pretreated with GA. The ability of the LPEI-max spacer to bind a protein probe to the polyHEMA surface was negligible: similar to the non-specific adsorption observed for the untreated polyHEMA film controls (FIG. 4A, left).

### Example 4

### Trypsin Immobilization to the PolyHEMA Film

In a set of recognition studies, an in vitro competence ELISA analysis was performed to examine the ability of a polyHEMA immobilized trypsin to capture A1AT. Based on the studies with the various types of spacers, described above, LPEI, NHS-^{3K}PEG-NHS and a mixture of NHS-^{3K}PEG-NHS with NHS-^{2K}PEG were tested. Thus, trypsin was reacted with dry polyHEMA films pre-grafted with either: (a) 7 mg/ml of LPEI (25 kDa); (b) 100 mg/ml of NHS-^{3K}PEG-NHS, or (c) a mixture of NHS-^{3K}PEG-NHS and NHS-^{2K}PEG (10+100 mg/ml). The films were incubated (PBS pH 5, 4° C., overnight) with 100 μg/ml trypsin followed by a Tween-20 (0.1% w/v in PBS) rinse to remove unbound trypsin. Nonspecific binding was blocked with BSA (1% w/v in PBS).

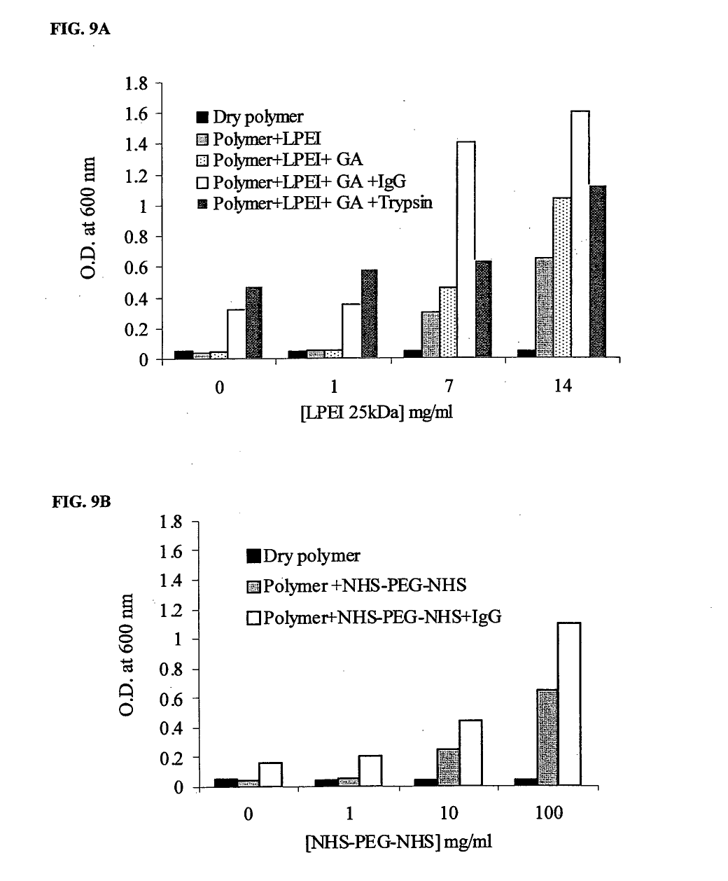

Film transparency of the spacer-containing polyHEMA films after trypsin conjugation was assessed, in dry or hydrated (3 pH values: 2.5, 5 and 7.5) states, by measuring their transmittance (optical density) at a wavelength range of 600 nm (within the range of 400-650 μm, relevant to A1AT capturing by the immobilized trypsin) in a microplate reader. The results show that grafting of the various substitutes reduced the films transparency (absorbance was increased). A full set of treatments of the polyHEMA film (LPEI—25 kDa, trypsin, or IgG) caused a 4.7-fold increase (from 0.3 to an O.D. value of 1.65) compared with the absorbance of a plain polyHEMA film in a dry state (FIG. 9). Replacing LPEI with the NHS-3 KPEG-NHS spacer improved the transparency by 33%. A full set of treatments of the polyHEMA film (NHS-3 KPEG-NHS, trypsin or IgG) led to an O.D. value of 1.1.

### Example 5

### A1AT Detection by the Immobilized Trypsin

The study was performed in a 96-well microplate containing 100 μl/well of polyHEMA-LPEI (25 kDa) films using samples onto which trypsin was immobilized, as detailed in Example 4. Non specific binding was blocked with BSA. After the addition of increasing concentrations (0-40 μg/ml) of A1AT (50 μl/well) the plate was incubated (60 min, 37° C.) and subsequently rinsed 3 times with PBS. The first antibody, rabbit polyclonal anti human A1AT, (0-40 μg/ml) was added to each well, followed by a similar incubation and PBS rinse. The HRP conjugated second antibody (1:5000 in PBS) was then added to the wells and the plate was rinsed and reacted with TMB reagent (in citrate buffer, pH 5 to a final volume of 100 μl/well). The reaction was stopped with H₂SO₄ 1M (100 μl/well). Color intensity was measured in a microplate reader at 450 nm.

As detailed above, ELISA analysis of the captured A1AT was conducted by rabbit anti-A1AT (the detecting antibody), followed by quantification with HRP-conjugated anti-rabbit IgG (the 2nd antibody). The results are summarized in FIG. 8, which demonstrates that when NHS-PEG-NHS was used to link trypsin to the surface of the polyHEMA, A1AT was captured significantly better compared with the binding accomplished when LPEI was used as a spacer arm. Overall, the use of a spacer for trypsin immobilization was crucial for A1AT capturing. The inhibitor was hardly recognized when trypsin was conjugated directly, i.e., with no spacers, to the surface of the polyHEMA film (data not shown).

### Example 6

### The SMSA Glass Slide Recognition Platform and its Spacer Arms

Three types of spacer arms, containing N-hydroxy-succinimide leaving group, were used in the SMSA glass slides experiments: (a) the bifunctional NHS-carbonate-NHS; (b) the bi-functional NHS-^{3k}PEG-NHS, or a mixture of NHS-^{3k}PEG-NHS with the mono-functional NHS-^{2k}PEG; and (c) heterobifunctional MAL-^{5k}PEG-NHS. The monofunctional NHS-^{2k} PEG served as a control spacer (is not expected to bind proteins after reaction with the glass surface). The grafting of the N-hydroxy-succinimide leaving group spacers onto the surface of the SMSA glass slides chemical formulas of the three spacers are shown is as follows:

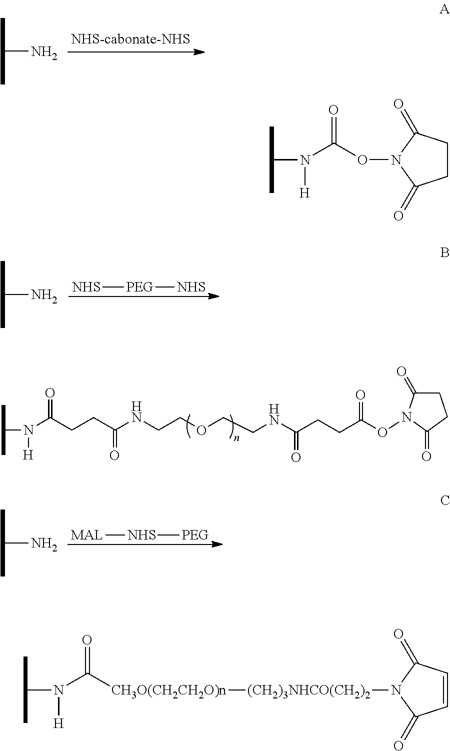

Grafting the SMSA glass slide with the spacer arms was performed as follows:

Increasing concentrations (3-30 mM) of the spacers (50 μl) were reacted with the SMSA glass slides (25° C., 1 h), followed by a PBS rinse to remove physically adsorbed spacers residues.

The ability of the SMSA-NHS-^{3K}PEG-NHS products to interact with proteins was examined by incubating the spacer arm-grafted platforms with 50 μL (10 μg/ml) of the Alexa Fluor⁵⁵⁵ labeled ovalbumin or with 25 or 100 μg/ml of the Alexa Fluor⁵⁶⁸ labeled IgG in PBS, pH 7.4 at 25° C. for one hour. Unbound protein was be removed by Tween-20 (0.1% w/v in PBS) rinse. Binding fraction (%) of the Alexa Fluor⁵⁶⁸ labeled IgG to the NHS-^{3K}PEG-NHS modified platform was determined in saturated system (with 25 μg/ml of the IgG). Fluorescent intensity of the SMSA substrate slides platform was measured by the GenePix Pro 4000 scanner (Axon Instruments, Inc., USA).

The ability of the SMSA-MAL-^{5K}PEG-NHS products to interact with proteins was examined by incubating the spacer-grafted platforms with 50 μL of reduced TRITC-conjugated AffiniPure F (ab′)₂ fragment of the goat anti-mouse IgG (H+L) (25-100 μg/mL), dissolved in PBS, pH 6.5 at 37° C. for one hour. Unbound protein was removed by Tween-20 (0.1% w/v in PBS) rinse. Fluorescent intensity of the SMSA substrate slides platform was measured in Microarray Axon scanner.

The role of spacers in protein binding to the SuperAmine 2 glass surface was examined, using Alexa Fluor⁵⁵⁵ labeled OVA as a recognizable protein model, NHS-carbonate-NHS (a short spacer arm model) and NHS-^{3K}PEG-NHS (a long spacer arm mode). The findings are summarized in FIG. 12, which shows that, NHS-carbonate-NHS was non-efficient in the specific binding of the OVA, apparently due to its high non-specific adsorption to the naked glass surface. On the other hand, concentration-dependent binding of the ovalbumin (OVA) was observed when the surface of the glass slide was modified with increasing amounts of the NHS-^{3K}PEG-NHS spacer.

### Example 7

### A1AT Detection by Immobilized Trypsin on the Glass Slide and the Selection of a Spacer Mixture

Mixtures of the bi-functional spacer, NHS-^{3k}PEG-NHS and the mono-functional spacer, NHS-^{2k}PEG were tested for their influence on the efficiency on the A1AT recognition by immobilized trypsin (100 μg/ml) to the glass slide.

Trypsin attachment to the surface of the SMSA glass slides modified with NHS-^{3k}PEG-NHS (30 mM) was performed by incubation (37° C., one hour) with trypsin (100 μg/ml in PBS, pH 5). Unbound trypsin will be removed by Tween-20 (0.1% w/v in PBS) rinse. Nonspecific binding was blocked by BSA (1% w/v in PBS for one hour at 37° C.).

Trypsin attachment to the surface of the SMSA glass slides modified with MAL-^{5K}PEG-NHS (20 mM) was performed by incubation (37° C., one hour) with trypsin (100 μg/ml in PBS, pH 5) bearing sulfhydryl groups. Unbound trypsin was removed by Tween-20 (0.1% w/v in PBS) rinse. Nonspecific binding was blocked by BSA (1% w/v in PBS for one hour at 37° C.).

The amount of recognized A1AT was analyzed by sandwich ELISA, using rabbit anti-A1AT in SGF. Further, detection of the A1AT-anti-A1AT complex was performed by two independent assays. The first employed Alexa Fluor⁶⁴⁷ conjugated anti-rabbit IgG, while the second employed HRP-conjugated anti-rabbit IgG. Fluorescence was measured at 635 nm (ex) and 660 nm (em) for the first detection method. Color intensity was measured at 450 nm for the later.

The results, summarized in FIG. 13, show that the fluorescent signal was much higher (SNR of 6-11, depending on the concentration of the A1AT) when a mixture of NHS-^{3k}PEG-NHS and NHS-^{2K}PEG was used, compared with the use of NHS-^{2K}PEG only. FIG. 13B shows that the use of spacer was crucial for the detection process.

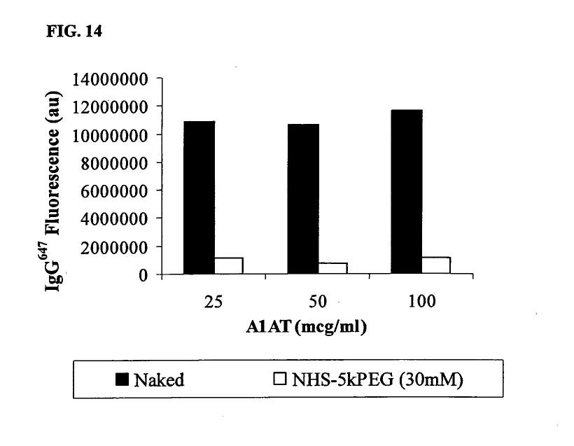

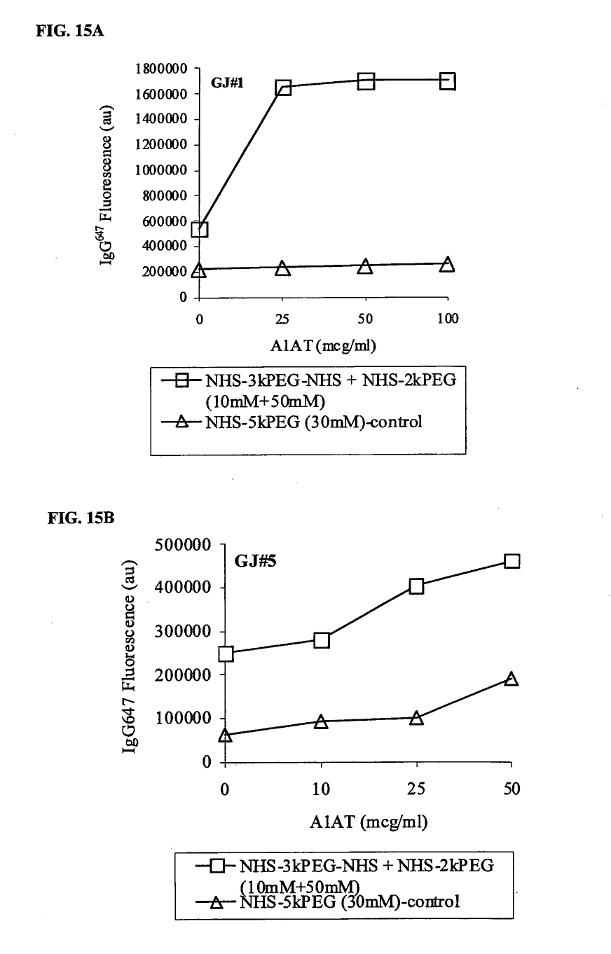

Another spacer tested, unsuccessfully, was the bi-functional NHS-carbonate-NHS, which was compared, under the same conditions mentioned above, to the mono-functional PEG spacer, NHS-^{5k}PEG spacer. After incubation with increasing concentrations of A1AT in SGF, rabbit A1AT antibody labeled with Alexa Fluor⁶⁴⁷ was added and fluorescence was measured (ex. 635 nm, em. 660 nm). FIG. 14 shows that not only that the NHS-carbonate-NHS did not contribute to a specific reaction between the immobilized trypsin and A1AT, the use of NHS-^{5k}PEG spacer reduced the interaction profoundly (10 to 14-fold) and ensured specific recognition of the MAT (see FIGS. 13 and 15).

In this part of the study the ability of the immobilized trypsin to detect A1AT in gastric juice aspirated from seven healthy human volunteers was tested (the results of two are shown in FIG. 15). Because the pH of the samples was commonly low (1-4), they were pre-buffered with a carbonate buffer solution (NaHCO₃, 3.57 mM; citric acid, 0.8 mM) by diluting them 10-fold (based on the findings by the Israelitisches Hospital, Germany). In separate studies, increasing concentrations of A1AT were added to the buffered gastric juice. To the resulted products rabbit anti-A1AT IgG was added, followed by anti-rabbit IgG labelled with Alexa Fluor⁶⁴⁷. Similar to the results shown in FIG. 13A, fluorescence was profoundly higher (with a SNR: 4 to 7) when using a mixture of bi- and mono-functional spacers: NHS-^{3K}PEG-NHS and NHS-^{2K}PEG for trypsin immobilization, compared with the fluorescence obtained with glass slide covered with NHS-^{2K}PEG spacer.

It is noteworthy that low concentration of the A1AT were detected in 4 (out of 7) gastric juice specimens.

### Example 8

### Trypsin Modification

To increase the spacer efficiency, a hetero-bi-functional product was be used instead of the homo-bi-functional NHS-3 KPEG-NHS spacer. The specific spacer used is N-[(3-maleimido-1-oxopropyl)aminopropyl-ω-(succinimidyloxy carboxy) polyoxyethylene glycol 5,000 (MAL-5KPEG-NHS). To conjugate it to trypsin, the protein must be modified to allow a reaction with the maleimide end of the spacer. For that purpose a thiol (—SH) group was introduced into the trypsin molecule. Thus, amine groups of trypsin and the proteinecious probe, fluorescently-labeled OVA555, were substituted with a thiol group, by using N-succinimidyl-3-(2-pyridyldithio)propionate (SPDP) and dithiothreitol (DTT).

In order to obtain covalent binding of the trypsin to the heterobifunctional MAL-^{5K}PEG-NHS spacer, the sulfhydryl groups were chemically attached to the proteins. The modification was performed according to the previously described procedure with our modifications. For this trypsin (0.1 mg) was reacted with 200-fold molar excess of the SPDP for 30 min at r.t. Then, unreacted SPDP was separated from the trypsin-SPDP complex with the aid of the Microcon YM-10 ultrafiltration device having low-binding, hydrophilic cellulose membrane with cut-off of 10 kDa (Millipore, Billerica, Mass., USA) by centrifugation for 30 min, 14000 rpm at 20° C. Collected trypsin was then reduced with 1 mM DTT (PBS, pH 7.4, 2 mM EDTA) by incubation for 30 min at r.t under N₂ atmosphere. Free DTT was separated from the modified protein with the aid of the Microcon YM-10 ultrafiltration device by centrifugation for 30 min, 14000 rpm at 20° C. Collected modified trypsin was resuspended in PBS (pH 6.5) and stored at −20° C. until used.

The capability of the MAL-^{5K}PEG-NHS spacer to prevent non-specific binding of the proteins, while specifically bind thiol-modified proteins was examined using Alexa fluor labeled OVA⁵⁵⁵ and thiol-modified OVA⁵⁵⁵ after reaction with the SMSA glass slide. Fluorescent was measured fluorometrically (ex. 635, em. 660). FIG. 16, showing the mean values of two experiments, clearly shows that MAL-^{5K}PEG-NHS was able to prevent non-specific binding of the OVA⁵⁵⁵ almost 5-fold better than naked glass. OVA⁵⁵⁵-SH also was bound specifically to the spacer-modified SMSA with a SNR of 2 to 11 compared with OVA⁵⁵, depending on the spacer's concentration.

Trypsin modification was performed according to the 2,2′-dithiodipyridine (DTDP) method described above. According to this method 2 SH groups were attached to each trypsin molecule. Elevating this number was found to interfere with the A1AT recognition (as was observed when 7 thiol groups were inserted).

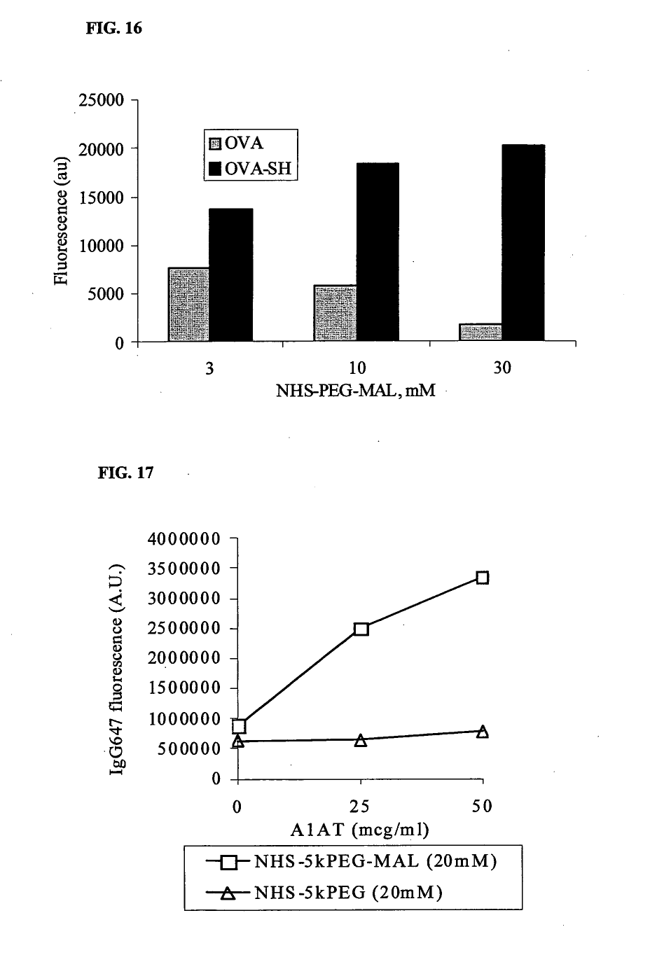

Detection of increasing amounts of A1AT by the new detecting platform was measured in SGF buffered with carbonate buffer solution, assayed by the sandwich ELISA method, employing rabbit anti-A1AT IgG and Alexa Fluor⁶⁴⁷ conjugated anti-rabbit IgG. Similarly to the results shown in FIG. 13A, fluorescence was much higher (SNR of 7) when MAL-^{5K}PEG-NHS was used as a spacer for immobilization of trypsin-SH, than glass slide coated with NHS-^{2K}PEG (FIG. 17).

### Example 9

### Development of an Immuno-Recognition Detecting Platform Conjugating Alexa Fluor⁵⁶⁸ Labeled IgG to SupeAmine 2 Glass Slide

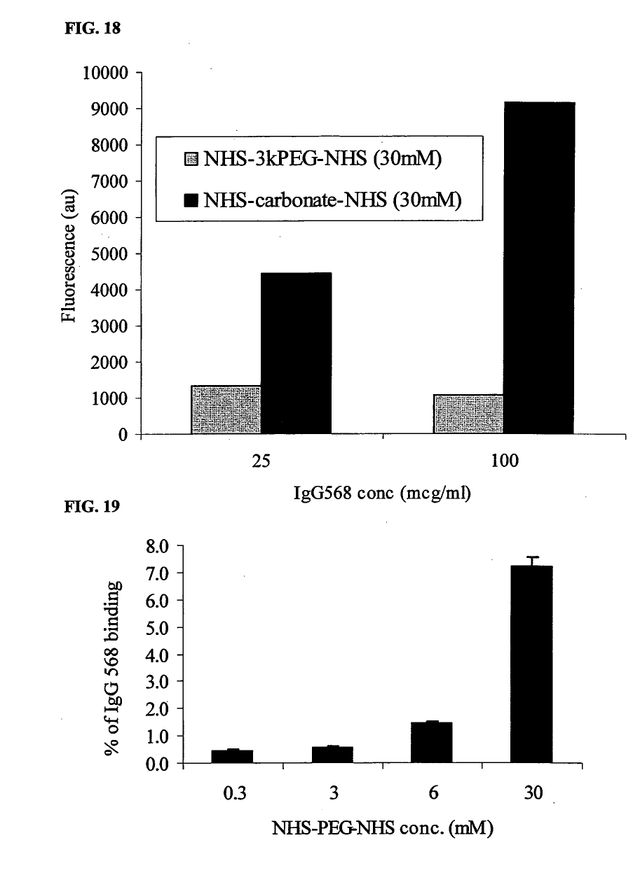

In order to develop a wide-ranging detecting platform based on immunological recognition reactions Alexa Fluor⁵⁶⁸ labeled IgG was attached to the SupeAmine 2 glass slide. This was checked with the short homo-bi-functional spacer NHS-carbonate-NHS and the longer homo-bi-functional spacer NHS-^{3K}PEG-NHS. Naked glass surface and NHS-^{2k}PEG-modified surface served as controls for the determination of the non-specific binding and calculation of IgG specificity. It was found that, in two concentrations, IgG was bound more efficiently to the glass slide than the binding with the longer spacer (FIG. 18). This could be due to steric interference and possibly low accessibility of the IgG which is 7-fold larger in mass than trypsin. The attachment of the protein to the SMSA glass slide was dependent on the surface density of the spacer molecules. A 4-fold increase (0.4-7% of binding, calculated on the basis of the initial amount of IgG employed) in the binding was observed when spacer amount varied from 0.3-30 mM (FIG. 19).

### Example 10

### Conjugating Reduced IgG (Fab′)₂ to SupeAmine 2 Glass Slide

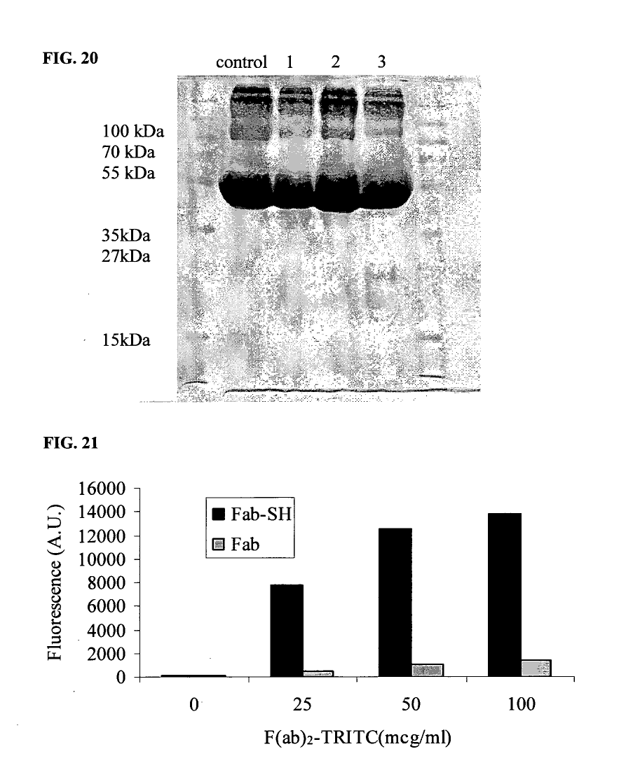

Because of the asymmetric nature of IgG, it may lose activity upon covalent attachment to solid surfaces. Therefore, in order to develop a high performance immunosensor for the detection of target molecules, orientation of the immobilized antibodies was carried out. Thus, we examined the immobilization of a reduced (Fab)₂ fragment of IgG into a F(ab) fragment to the MAL-^{5K}PEG-NHS spacer, which was previously covalently bound to the surface of the SMSA glass slide. For this purpose the tetramethyl Rhodamine isothiocyanate (TRITC)-conjugated (Fab)₂ fragment of the IgG was reduced with DTT into intact monovalent Fab fragments. The integrity of the resulted antibody fragments was analyzed by the SDS-PAGE electrophoresis (FIG. 20).

In order to obtain covalent binding of the F(ab′)₂ fragment of the IgG to the heterobifunctional MAL-^{5K}PEG-NHS spacer, the S—S bonds present in the hinge region of the F (ab′)₂ were reduced in the presence of 2.5 mM DTT. For this, 250 μl containing 0.06 μg of the F(ab′)₂ were incubated with 50 μl of the DTT (f c. 0.5 mM in PBS, pH 6.5 containing 2 mM EDTA) for 30 minutes at room temperature. under N₂ atmosphere. Free DTT was separated from the modified protein with the aid of the Microcon YM-10 ultrafiltration device by centrifugation for 30 min, 14000 rpm at 20° C. Collected modified protein was resuspended in PBS (pH 6.5) and stored at 4° C. until used. The integrity of the reduced F(ab′)₂ was determined by non-reduced SDS-PAGE (12.5%) using Vertical Gel Electrophoresis System (Bio-Rad). The gel was stained with Coomassie brilliant blue R250. Control experiments were run using unreduced mAb F(ab′)₂.

Based on the appearance of the disintegrated heavy- and light-chain portions of the (Fab′)₂ in the reduced (2.5 mM DTT) protein, (Fab′)₂ was reduced with 0.5 mM DTT. The resulted reduced (Fab)₂ fragment and a non-reduced, intact (Fab)₂ were reacted with SMSA glass slide, pre-treated with MAL-^{5K}PEG-NHS. The results shown in FIG. 21 demonstrate that reduced (Fab′)₂ binds specifically to the spacer-modified glass slide, while intact (Fab′)₂ show almost no binding (SNR of reduced (Fab′)₂ to intact (Fab′)₂: 10-19 depending on the concentration of the (Fab′)₂).

1. A device for in-vivo detection of a biomarker in the gastrointestinal system, the device comprising:
a housing comprising an optical window and enclosing a light receptor and a light source for illuminating in-vivo through said optical window;
wherein an external surface of the optical window is coated by a polymer, said polymer having a recognition factor immobilized thereon via a spacer, and wherein the light receptor is configured to detect light changes on the illuminated surface of the optical window.

2. The device according to claim 1, wherein said light receptor is a photodetector covered by high pass- or a notch filter configured to detect fluorescent changes on the illuminated surface of the optical window.

3. A device according to claim 1, wherein the light sours is one or more LED.

4. A device according to claim 1, wherein the light receptor is an imager.

5. A device according to claim 1, wherein light receptor is a CMOS detector or imager.

6. The device according to claim 1, wherein said polymer is polyHEMA.

7. A device according to claim 1, wherein the spacer is a PEG based spacer selected from the group consisting of O,O′-bis[2-(N-succinimidyl-succinylamino)ethyl]polyethylene glycol (NHS-3 KPEG-NHS) and a combination of NHS-3 KPEG-NHS and O—[(N-succinimidyl)succinyl-aminoethyl]-O′-methylpolyethylene glycol (NHS-2 KPEG).

8. A system for in-vivo detection, the system comprising:
a device according to claim 1;
a transmitter to transmit data from the light receptor;
a receiving system to receive transmitted signals; and
a display to display indication of the presence of a marker in vivo.

9. (canceled)

10. (canceled)

11. (canceled)

12. (canceled)

13. A method for the in vivo detection of the presence of a specific cancer biomarker in the gastrointestinal system of a subject comprising the steps of:
orally administering a device according to claim 1 to the subject;
contacting the orally administered device with a detectable labeled binding agent that binds specifically to the biomarker or contacting the orally administered device with a first binding agent that binds specifically to the biomarker and a second detectable labeled binding agent that binds specifically to the first binding agent;
wherein the presence of a bound label as detected by the light receptor is indicative to the presence of said specific biomarker in the gastrointestinal system of the subject.

14. (canceled)

15. (canceled)

16. (canceled)

17. (canceled)

18. (canceled)

19. (canceled)

20. A polycarbonate film coated by a recognition platform that binds to a specific cancer biomarker; wherein the recognition platform comprises a polymer having a recognition factor immobilized thereon via a spacer.

21. (canceled)

22. The polycarbonate film of claim 20, wherein the spacer molecule is a PEG based spacer selected from the group consisting of O,O′-bis[2-(N-succinimidyl-succinylamino)ethyl]polyethylene glycol (NHS-^{3K}PEG-NHS) and a combination of NHS-^{3K}PEG-NHS and O—[(N-succinimidyl)succinyl-aminoethyl]-O′-methylpolyethylene glycol (NHS-^{2K}PEG).

23. A glass slide that binds to a specific cancer biomarker; wherein the glass slide comprises a recognition factor immobilized thereon via a spacer.

24. The glass slide of claim 23, wherein the glass slide is Super Mask™ SuperAmine 2 (SMSA).

25. The glass slide of claim 23, wherein the spacer molecule is a PEG based spacer selected from the group consisting of O,O′-bis[2-(N-succinimidyl-succinylamino)ethyl]polyethylene glycol (NHS-^{3K}PEG-NHS) and a combination of NHS-^{3K}PEG-NHS and O—[(N-succinimidyl)succinyl-aminoethyl]-O′-methylpolyethylene glycol (NHS-^{2K}PEG).

26. The device according to claim 1, wherein said optical window is made of polycarbonate.
